(19) United States
(12) Patent Application Publication
(10) Pub. No.: US 2024/0408325 A1
(43) Pub. Date: Dec. 12, 2024

(54) HANDHELD MEDICAL DEVICE WITH IMPROVED DELIVERY OF AEROSOL PARTICLES

(71) Applicant: ALEXZA PHARMACEUTICALS, INC., Fremont, CA (US)

(72) Inventors: David Koji HASEGAWA, Fremont, CA (US); Peter HOLST, Fremont, CA (US); Dmitriy PETROVYKH, Fremont, CA (US)

(73) Assignee: ALEXZA PHARMACEUTICALS, INC., Fremont, CA (US)

(21) Appl. No.: 18/743,451

(22) Filed: Jun. 14, 2024

Related U.S. Application Data
(63) Continuation of application No. PCT/US2022/052907, filed on Dec. 14, 2022.
(60) Provisional application No. 63/289,605, filed on Dec. 14, 2021.

(51) Int. Cl. A61M 11/04

(52) U.S. Cl. CPC A61M 11/042; A61M 2202/0007; A61M 2205/36; A61M 2206/10

(57) ABSTRACT

A handheld medical device with internal structures to improve the delivery of aerosol particles and its uses. The structures include airflow diverting structures, airflow straightening structures and/or internal walls defining an angle.

### FIELD

The present methods and devices relate to the field of aerosol drug delivery devices, specifically to the field of devices for the precise administration of condensation aerosols.

### BACKGROUND

Many drug condensation aerosol drug delivery devices are currently known. Most of them fall under the electronic cigarette type, where a liquid is heated to form an aerosol that is then inhaled, or the source of the aerosol is a plant product. However, those devices are not suitable to administer drugs that need precise dosing. For this purpose, it is preferable to use devices where the dose of the drug to be aerosolised is measured with high precision, such as by precisely depositing it into a surface (substrate) to be heated.

Further information on condensation aerosols, formation of condensation aerosols, drug delivery devices, drug composition film thickness (or solid drug film thickness), substrate area, aerosol particle size distribution (aPSD), mass median aerodynamic diameter (MMAD), analysis of drug-containing aerosols, other analytical methods, vaporization temperature control or drug foil substrate design can be found, inter alia, in the description and examples of WO2019152873, WO2017189883 or WO2016145075 which are incorporated by reference.

It is required that the dose of the drug that leaves such devices (i.e. the emitted dose, ED) is consistent each time a device is actuated so that the dosing is accurate.

In WO2019152873 (WO'873) a disposable cartridge with a drug coated on an electrically heated drug foil substrate is described. The disposable cartridge can be connected to a handheld controller which includes the electronics and electrical components, including the electricity source. Between the air inlet and the drug foil substrate there is a perforated bulkhead.

In FIG. 17 of document WO'873 obstructions introducing turbulence to achieve more uniform heat loss of the drug foil substrate are disclosed. These elements protrude from the surface of the airway upstream and in the vicinity of the drug foil substrate. These elements can be posts, bumps and so forth. The elements in FIG. 17 of document WO'873 are only attached to one of the walls of the airway. Generally, obstruction elements would be positioned upstream of the drug foil substrate to minimize deposition of aerosol particles on them.

In FIG. 5 of WO2017189883 a device with rear air inlet (512) where the airflow (508a) is diverted using an air by-pass (508) is shown. In this device, the optional one or more openings, holes, or slits (509) are similar to the perforated bulkhead of WO'873. Before approaching the drug surface (502), the airflow needs to turn towards it, but no specific feature to turn the airflow is indicated.

In FIG. 1B of WO2005120614 (WO'614) a device having two airways, one airway (20) above the structure 22 and a second airway (21) going below the structure 22 and passing through the holes, as indicated by arrows 23, is offered as a solution to reduce aerosol deposition from upstream support 24 onto downstream supports 27 in multidose devices.

In FIG. 2F of WO'614 a device including multiple doses is shown. The airflow (36) enters the airway (30) and passes through a plurality of drug supports (40) and then leaves the device through the mouthpiece (45) parallel to the entering direction. FIGS. 2A-2E of WO'614 show different embodiments where the airflow passes through the drug supports (40).

The examples in document WO'614 relate to multidose devices including 25 doses of 50 μg of fentanyl. The problem of aerosol deposition on downstream supports in multidose devices is addressed in this document. Each of the 25 doses is provided by heating 25 individual metal foils (acting as drug foil substrates). One metal foil is heated in each dosing to generate an aerosol, but part of it deposits back on the remaining unused metal foils. Therefore, the amount of drug available to generate the aerosol on the remaining unused metal foils increases depending on the amount deposited by the previous doses. This causes that the dose that leaves the device (i.e. the emitted dose) may vary uncontrollably with time depending on the amount of drug that is deposited back with the previous doses.

In FIG. 1C of WO2008112661 a device having two air inlets (154 and 156) is disclosed. In said device the substrate (158) includes a plurality of holes (not shown) fluidly connecting the first airway (164) and the second airway (166) and the air is forced to turn from one airway to another via the holes in the substrate (158).

In WO2016145075 a way of reducing aerosol deposition on the internal housing is disclosed including the use of antistatic materials on the internal walls of the device or manufacturing the full device using antistatic materials.

Even with the solutions available, some drugs have a very broad distribution of emitted dose (ED) (see Comparative Example 2) and in some cases the drug adheres to the internal walls of the housing of the condensation aerosol generating devices, e.g. apomorphine as shown in FIG. 1 and Comparative Example 1.

Further, it is preferable to use the lowest temperature possible to generate the aerosol in order to avoid any discomfort to the user due to the increased temperature of the inhaled aerosol and to reduce the degradation that the aerosolized products suffer at higher temperatures. (see Comparative Examples 3 and 4).

Thus, there is a need in the art for a device capable of administering a reproducible (with narrow distribution of emitted dose) and accurate dose of a condensation drug aerosol with high aerosol purity in order to increase the reproducibility of the emitted dose and thus improve the therapy.

### SUMMARY

In one aspect of the disclosure is a handheld medical device suitable to generate a drug condensation aerosol by thermal vaporization of a drug including:
a. an airway (203) defined by internal walls;
b. an air inlet (220) at one end of the airway (203);
c. an air outlet (202), configured as a mouthpiece, at another end of the airway (203);
d. a drug foil substrate (205) having an impermeable surface, with or without perforations, placed within the airway (203);
e. a drug foil substrate support (204);
f. a solid drug film (207) coated on the drug foil substrate (205);
g. an aerosol axis (223) defined by the geometrical center of the solid drug film (207) and the geometrical center of the air outlet (202), where the downstream direction (D_{A}) goes from the geometrical center of the solid drug film to the geometrical center of the air outlet (202) and the upstream direction (U_{A}) is the opposite direction;
h. a drug foil substrate heating circuit to vaporize at least part of the drug in the solid drug film (207) to produce condensation aerosol particles, where the drug foil substrate heating circuit includes at least one battery (101), and
i. aerosol particle delivery modifiers selected from at least one of:
   I. one or more airflow diverting structures (224), which include a structure placed upstream of the drug foil substrate (205) including a plurality of channels (224CH), where the center lines of the channels define an angle β from 100° to 170° with the ray of the aerosol axis (223) in the upstream (U_{A}) direction measured in the sector containing the air inlet (220);
   II. one or more airflow straightening structures (225), which include a structure (225) placed upstream the drug foil substrate (205) including a plurality of channels (225CH), where the centerlines of the channels define an angle α from −10° to 10° with the aerosol axis (223);
   III. internal walls including two internal opposite walls (222, 222a, 222b) which define an angle δ of 5° to 20° in at least 80% of its portion included within the projection (Pf) of the drug foil substrate (205) on the aerosol axis (223); where the vertex of said angle δ is located in the upstream (U_{A}) direction; and
   IV. one or more airflow directing structures (226), which include a structure (226) placed upstream the drug foil substrate (205) including at least two sets of channels (226CHL and 226CHR), one set at each side of the Z-plane, where the centerlines of each set of channels (226CHL and 226CHR) define an angle γ from 5° to 20° with the Z-plane; where the vertex of said angle γ is located upstream the drug foil substrate.

Another aspect is a disposable cartridge (200) suitable to be connected to a handheld controller (100) which includes features a) to g), and i) according to the previous aspect.

Another aspect is a device or disposable cartridge for use in therapy according to any of the preceding aspects, where when the drug in the solid drug film (207) is:
A. loxapine or its pharmaceutically acceptable salts, the condition or episode is agitation, including:
   i. rapidly control mild to moderate agitation in adults with schizophrenia or bipolar disorder, or
   ii. acute agitation associated with schizophrenia or bipolar disorder in adults;
B. alprazolam, estazolam or its pharmaceutically acceptable salts, the condition or episode is epilepsy, where epilepsy includes seizures;
C. fentanyl or its pharmaceutically acceptable salts, the condition or episode is breakthrough pain;
D. zaleplon, almorexant or its pharmaceutically acceptable salts, the condition or episode is a sleep disorder including:
   i. middle of the night awakening, or
   ii. middle of the night insomnia;
E. apomorphine, pergolide, ropinirole, pramipexole, or its pharmaceutically acceptable salts, the condition or episode is Parkinson's disease, off-episodes in Parkinson's disease, and/or idiopathic Parkinson's disease;
F. granisetron, ondansetron, palonosetron or its pharmaceutically acceptable salts, the condition or episode is:
   i. nausea,
   ii. vomiting or
   iii. cyclic vomiting syndrome;
G. nicotine or its pharmaceutically acceptable salts including nicotine meta-salicylate, the condition or episode is nicotine craving and/or effecting cessation of smoking; or
H. ropinirole, pramipexole, or rotigotine the condition or episode is restless legs syndrome.

### Definitions

Within the present disclosure, the following terms are used with the following meanings.

“Handheld medical device” is a device which can be hold and operated with one hand. The handheld medical device includes all the elements required to be operated in one single device or more than one device, e.g. into two devices, such as a disposable cartridge (200) and a handheld controller (100).

“Disposable cartridge” is understood to be a single dose or multidose cartridge including a drug coated on a drug foil substrate, an air inlet that may or may not be connected to a handheld controller, an airway (203), an air outlet configured as a mouthpiece and connectors to electrically connect it to the handheld controller (100). The disposable cartridge is attached to a handheld controller in order to be operated.

“Handheld controller” is a reusable device including at least one battery, connectors, electrical parts, and electronics suitable to heat the drug foil substrate once a disposable cartridge has been attached to it.

“Electrical connections” are means to transfer electricity from a battery to a drug foil substrate, a microcontroller or a memory.

“Data connections” are means to transfer data between a microcontroller and any sensor, detector, or memory, where the sensor or detector and memory are in the disposable cartridge and/or the handheld controller.

“Battery” means a device for storing energy in chemical compounds capable of generating an electrical current. It can be a rechargeable battery, such as nickel-metal hydride, lithium-polymer battery, or the like (see WO2019152873 for further details on rechargeable batteries); or a single use battery, such as a dry cell battery, alkaline batteries, silver cells batteries, zinc-air batteries, lithium batteries, nickel oxyhydroxide batteries and the like. When the heating to vaporize a drug is chemical heating the battery is needed to start the chemical reaction and/or control the electronics of the device.

“Air inlet controller extension” is an extension of the airway of a disposable cartridge (200) in a handheld controller (100).

“Pneumatic sealing” is a piece, such a gasket or an O-ring, that helps joining two systems, e.g. ducts, and prevents air leakage.

“Airway” is defined by the device internal walls with an air outlet (202) configured as a mouthpiece at one end and one or more air inlets (220) at the other end. The one or more airflow diverting structures and the one or more airflow straightening structures are placed within the airway.

“Airflow” is the movement or flow of air through the airway from the air inlet (220) to the air outlet (202).

Since “airflow” and “airway” share the same space within the handheld medical device, they are referred to using the same reference number, 203.

“Airflow detector” is any sensor capable of detecting if the airflow is above a predetermined threshold value. The airflow detector can be of any kind, such as a differential pressure sensor, a thermistor, an air flow or sail switch, a hot wire anemometer, or vane anemometer.

“Internal height” of the device is the distance between two opposite points on the internal surface of the device crossed by the Z-axis or a line parallel to it. The internal height may be different across the device.

“Channels” means conduits through which the airflow is conveyed. Channels can have any cross section, e.g. round, triangular, square, rectangular. The corners of the channels can be square or round. The channels can be straight or curved.

When referring to the “minimum width” of a channel it is meant that the channel may have a range of widths along its length and the minimum width is within the specified range or value, but other widths can be larger. The same applies to “minimum height”, “minimum width to height ratio” or similar expressions.

“Center of the mouth of a channel” is the point at the center of an end (upstream or downstream) of a channel.

“Cavities” are openings upstream and/or downstream the channels. The cavities can extend above and/or below the channels. The cross section of the cavities can have any shape, e.g. round, triangular, square, rectangle, etc. The corners can be square or round.

“Centerline direction” is the path following the centerline.

“Angles of non-intersecting lines or axis”. Even if two lines, axis or a line and an axis are in different planes they define an angle. Said angle is measured by projecting the first line or axis on the plane of the second line or axis.

“Aerosol axis-air inlet plane” is the plane defined by the aerosol axis (223) and the center of the air inlet (220).

“Downstream” means in the direction of the airflow (223) or nearer to the air outlet (202).

“Upstream” means in the direction opposite to the airflow (223) or nearer to the air inlet (220).

“U_{A}” refers to the upstream edge of the aerosol axis (223)

“D_{A}” refers to the downstream edge of the aerosol axis (223).

“Central region” of the airflow diverting structures (224) and/or the airflow straightening structures (225) is a region around the center of such structure. “Central region that includes half of the channels” refers to a central region that includes half of the channels (if the number of channels in N, and N is uneven N/2 is rounded to the next integer) and the remaining channels are divided equally between both sides of the central region. “Central region that includes a third of the channels”, “Central region that includes a quarter of the channels”, and similar expressions are defined analogously.

“Drug foil substrate” is an impermeable surface where a drug is deposited to be heated and aerosolized. If the heating is electric, further information on electric heating can be found in WO2019152873, incorporated herein by reference. If the heating is chemical, it may be referred to as chemical heating pack, further information on chemical heating packs can be found in WO2004104492, incorporated herein by reference. The drug foil substrate defines a “drug foil substrate plane” which is preferably substantially parallel to the aerosol axis (223), i.e. defining an angle of −10 to 10°. The drug foil substrate may be planar or curved.

“The Z-plane” is the plane formed by the aerosol axis (223) and the Z-axis.

“The most downstream drug foil substrate point” is the point of the drug foil substrate (205) closest to the center of the air outlet. In case there is more than one point that could be considered the most downstream point, the closest to the aerosol axis is selected. “The most upstream drug foil substrate point” is the point at the edge of the drug foil substrate (205) crossed by the plane defined by the most downstream drug foil substrate point and the aerosol axis.

“The longitudinal axis of the drug foil substrate” is the axis defined by the most downstream drug foil substrate point and the most upstream drug foil substrate point.

“The lateral axis of the drug foil substrate” is the axis defined by the two further away points of the drug foil substrate (205) in the plane that contains the geometrical center of the solid drug film (207) and is orthogonal to the aerosol axis.

“Drug foil substrate support” is one or more elements to maintain the drug foil substrate within the airway (203). The drug foil substrate support can be placed within the airway (203), can include the walls defining the airway (203) and/or be partially outside the airway (203). The drug foil substrate support is referred to as number 204.

“Solid drug film” is a layer which includes a pure drug, two or more drugs, or one or more drugs in combination with additional components. Additional components can include, for example, pharmaceutically acceptable excipients, carriers, and the like.

“Solid drug film shape” may be in various configurations including, but not limited to, a trapezoidal shape, a crescent shape, a rectangular shape or a square shape (see WO2019152873, incorporated herein by reference, for further details). The shape of the drug coating area may follow a temperature map of the drug foil substrate during heating to allow for coating the solid drug film in reference to the regions of the drug foil substrate where the temperature profile is optimal for vaporization of the drug substance during heating of the drug foil substrate.

“Area density” in a two-dimensional object (such as the solid drug film (207)) is calculated as the mass per area unit.

“Condensation aerosol” refers to an aerosol that has been formed by the thermal vaporization of a solid drug film and subsequent cooling of the vapor, such that the vapor condenses to form particles.

“MMAD” refers to Mass Median Aerodynamic Diameter. The MMAD of an aerosol refers to the aerodynamic diameter for which half the particulate mass of the aerosol is contributed by particles with an aerodynamic diameter larger than the MMAD and half by particles with an aerodynamic diameter smaller than the MMAD.

“Next generation impactor” has the meaning given in US Pharmacopoeia (601) inhalation and nasal drug products: aerosols, sprays, and powders-performance quality tests.

“Multi-dose” refers the ability of an aerosol generating device to deliver more than one dose of the drug. This can be done by having more than one drug foil substrate (such as the devices disclosed in WO2005120614, incorporated herein by reference) or by repeatedly heating a drug foil substrate with remaining drug from the previous heating.

“Opposite walls” are walls, among the walls defining the airway (203) of the handheld medical device, facing each other. See FIGS. 13A and 13B and its description on how to determine if two walls face each other. If the walls are not straight, the same methodology as described above is applied but using the chords of the walls.

“Opposite walls angle δ” is defined by the intersection of the imaginary extension of angled part of the opposite walls around the drug foil substrate. If the opposite walls are curved the angle is defined by the chord of each wall connecting the curved extremes of the wall. The vertex of the imaginary extension of the opposite walls angle is placed upstream of the aerosol axis (U_{A})

“Antistatic materials” are materials with antistatic properties, i.e. materials that are slightly conductive (i.e. having a surface resistivity generally between 1×10⁷ and 1×10¹² (2/square) or make the surface slightly conductive and, thus, reduce or eliminate the build-up of static electricity. The antistatic material can be placed as coating on the internal walls of the device or can constitute all the material used to manufacture the airway. These antistatic properties can be introduced by coating the inner walls of the airway with conductive metals such as stainless steel and/or copper, and/or by applying a metallic tape (like a tape of copper) to the inner walls of the airway, the use of an antistatic spray (such as Staticide®, including quaternary ammonium compounds, coco alkylbis(hydroxyethyl)methyl, nitrates) on the internal walls of the airway, and/or by the use of antistatic plastics (such as polycarbonate, or polyamide (nylon) of the PermaStat® or PermaStat® plus brands) compounded with carbon based, metal-based or all polymeric alloys (with inherently dissipative polymers, such as Pebax® MH 2030 or PEBAX® MV 20 resins (both from Arkerma and made of flexible polyether and rigid polyamide) or Stat-Rite® family (from Lubrizol), other inherently dissipative polymers are polyamide/polyether block amides, polyether block amides based on PA6 or PA12 chemistry, ethylene ionomers, polyaniline polymer additives and liquid organic PEG-carboxylate esters. Commercial polymeric grades include Irgastat P (BASF), Entira (Dupont), Pelestat (Sanyo Chemical))) as the materials used to manufacture the walls of the device. Further information regarding the use of antistatic materials can be found in WO2016145075.

### BRIEF DESCRIPTION OF THE DRAWINGS

FIG. 1 illustrates previous devices.

FIG. 2A illustrates a disposable cartridge (200) which includes an airflow diverting structures (224), an airflow straightening structures (225) and straight internal opposite walls (222) forming an angle.

FIG. 2B illustrates the airflow diverting structures (224) and airflow straightening structures (225) of the device of FIG. 2A.

FIG. 3 is a calibration graphic of the emitted dose (ED) versus temperature for the prior art device shown in FIG. 1 using apomorphine HCl as drug.

FIG. 4A illustrates a schematic view of a prior art disposable cartridge.

FIG. 4B illustrates a schematic view of a disposable cartridge (200) with the air inlet (220) aligned with the aerosol axis (223).

FIG. 5A illustrates a handheld medical device including a disposable cartridge (200) and a handheld controller (100) where the air inlet (220) of the disposable cartridge is connected to the air inlet extension (103) of the handheld controller (100).

FIG. 5B illustrates a handheld medical device including a disposable cartridge (200) and a handheld controller (100) where the air inlet (220) of the disposable cartridge is directly open to the atmosphere.

FIG. 6A illustrates a schematic upper view of a disposable cartridge with lateral air entrance showing an airflow diverting structure (224).

FIG. 6B illustrates a schematic side view of a disposable cartridge with lower air entrance showing an airflow diverting structure (224).

FIG. 7 illustrates a schematic upper view of a disposable cartridge showing an airflow straightening structure (225).

FIG. 8 illustrates a schematic upper view of a disposable cartridge showing an airflow straightening structure (225) attached to an airflow diverting structure (224).

FIG. 9A illustrates a schematic upper view of a disposable cartridge with parallel straight walls.

FIG. 9B illustrates a schematic upper view of a disposable cartridge with parallel concave walls.

FIG. 9C illustrates a schematic upper view of a disposable cartridge with parallel convex walls.

FIG. 10A illustrates a schematic upper view of a disposable cartridge with angled straight walls.

FIG. 10B illustrates a schematic upper view of a disposable cartridge with angled concave walls.

FIG. 10C illustrates a schematic upper view of a disposable cartridge with angled convex walls.

FIG. 11A illustrates a schematic side view of disposable cartridges with parallel straight walls.

FIG. 11B illustrates a schematic upper view of a disposable cartridge with parallel concave walls.

FIG. 11C illustrates a schematic upper view of a disposable cartridge with parallel convex walls.

FIG. 12A illustrates a schematic upper view of a disposable cartridge with angled straight walls.

FIG. 12B illustrates a schematic upper view of a disposable cartridge with angled concave walls.

FIG. 12C illustrates a schematic upper view of a disposable cartridge with angled convex walls.

FIGS. 13A and 13B illustrate the relationship between opposite walls.

FIG. 14 illustrates a cross section of a 3D model a disposable cartridge.

FIG. 15A illustrates a different disposition of the drug foil substrate (205).

FIG. 15B illustrates a different disposition of the drug foil substrate (205).

FIG. 16A illustrates a 3D Hshape.

FIG. 16B illustrates a 3D ├ shape.

FIG. 16C illustrates a 3┤ shape.

FIG. 17A illustrates a close-up cross section of a 3D model of a disposable cartridge (200) showing details of an airflow straightening structure (225).

FIG. 17B illustrates a close-up cross sections of a 3D model of a disposable cartridge (200) showing details of an airflow diverting structure (224).

FIG. 18A illustrates a device as described in Device Configuration in the examples.

FIG. 18B illustrates a device as described in Device Configuration in the examples.

FIG. 18C illustrates a device as described in Device Configuration in the examples.

FIG. 18D illustrates a device as described in Device Configuration in the examples.

FIG. 18E illustrates a device as described in Device Configuration in the examples.

FIG. 18F illustrates a device as described in Device Configuration in the examples.

FIG. 18G illustrates a device as described in Device Configuration in the examples.

FIG. 18H illustrates a device as described in Device Configuration in the examples.

FIG. 18I illustrates a device as described in Device Configuration in the examples.

FIG. 18J illustrates a device as described in Device Configuration in the examples.

FIG. 18K illustrates a device as described in Device Configuration in the examples.

FIG. 19 illustrates a device with airflow directing structures (226).

FIG. 20A illustrates a device with airflow directing structures (226) in combination with internal opposite walls (222).

FIG. 20B illustrates airflow directing structures (226) having a rectangular shape (similar to angled rectangular fins).

FIG. 20C illustrates straightening structures (225) having an aerodynamic elliptical shape.

FIGS. 21A-21K illustrate specific embodiments of disposable cartridges according to FIGS. 18A-18K respectively.

FIG. 22A illustrates a velocity plot of a device according to Example 2.

FIG. 22B illustrates a velocity plot of a device according to Example 2.

FIG. 22C illustrates a velocity plot of a device according to Example 2.

FIG. 22D illustrates a velocity plot of a device according to Example 2.

FIG. 22E illustrates a velocity plot of a device according to Example 2.

FIG. 22F illustrates a velocity plot of a device according to Example 2.

FIG. 22G illustrates a velocity plot of a device according to Example 2.

FIG. 22H illustrates a velocity plot of a device according to Example 2.

FIG. 22I illustrates a velocity plot of a device according to Example 2.

FIG. 22J illustrates a velocity plot of a device according to Example 2.

### DETAILED DESCRIPTION

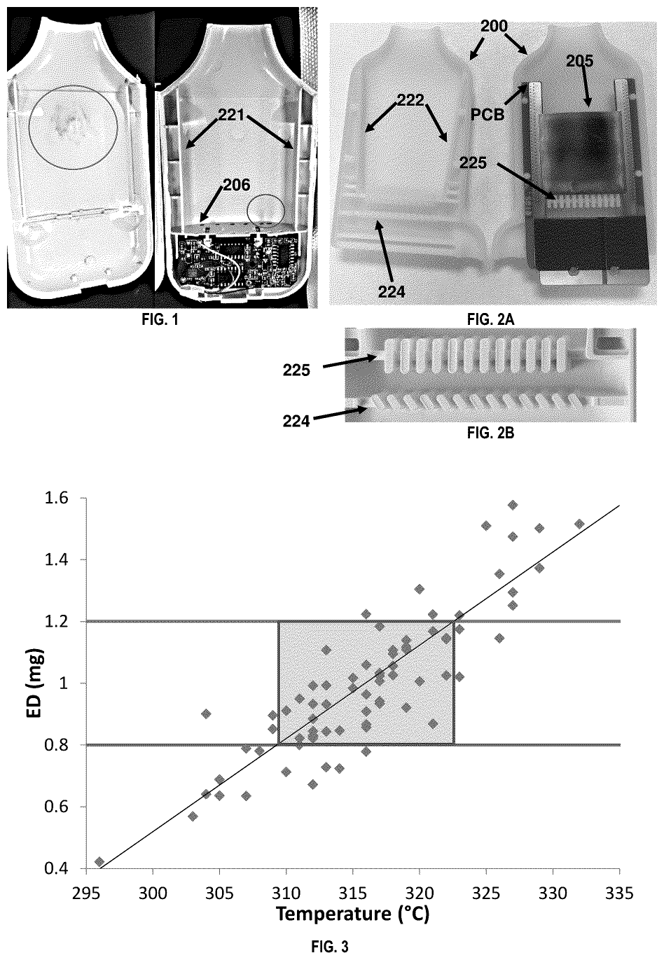

FIG. 1 illustrates a device suitable to generate a drug condensation aerosol by thermal vaporization similar to that marketed under the trademark Adasuve® for the inhaled delivery of loxapine but made of Permastat® (antistatic polycarbonate) material with a perforated bulkhead (206) and parallel straight internal opposite walls (221). The device has been opened after being used. A drug foil substrate (not shown) was coated with apomorphine HCl, which was vaporized by heat. Residual apomorphine HCl is found deposited in the middle of the wall near the mouthpiece (piece on the left, circled) and on the upstream-right part of the wall near the bulkhead (piece on the right, circled) after vaporization.

FIG. 2A illustrates a used opened housing of a device (in this case a disposable cartridge (200)) which includes one airflow diverting structure (224), one airflow straightening structure (225) and straight internal opposite walls (222) forming an angle. The half of the housing in the right includes a Printed Circuit Board (PCB) and an actuated drug foil substrate (205) (in this case previously coated with apomorphine HCl). No residual apomorphine HCl was observed on the internal walls of the housing.

FIG. 2B illustrates an enlarged view of the airflow diverting structure (224) and the airflow straightening structure (225) of the device of FIG. 2A.

FIG. 3 is the calibration graphic of the emitted dose (ED) versus temperature in the range 295-335° C. for the prior art device shown in FIG. 1 containing a drug foil substrate coated with 4 mg of apomorphine HCl for a target ED of 1 mg. 80-120% target ED is obtained within the 310-323° C. temperature range at an airflow of 30 L/min.

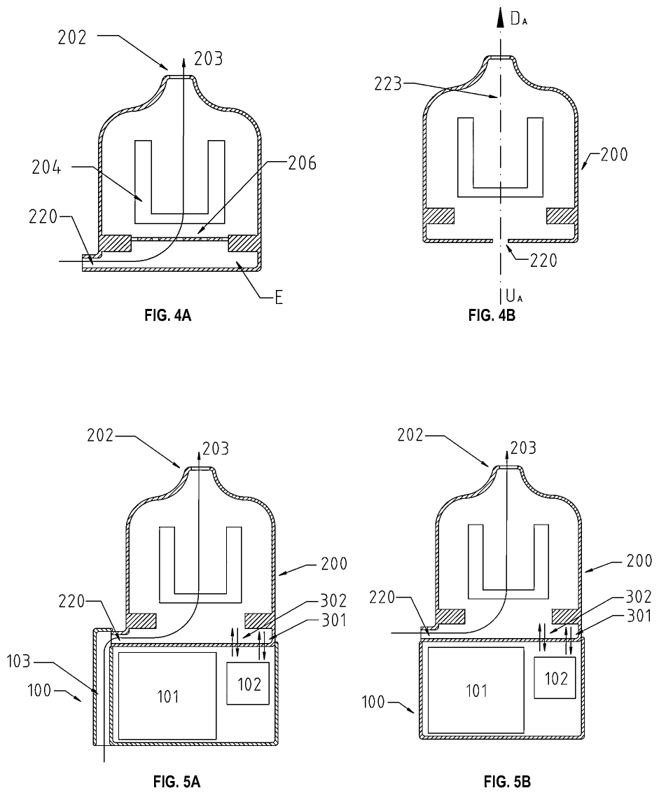

FIG. 4A illustrates a schematic view of a prior art handheld medical device where the airflow (203) enters from the air inlet (220) and passes through the perforated thin bulkhead (206) and the drug foil substrate support (204) and leaves the chamber through the air outlet configured as a mouthpiece (202). The drug foil substrate heating circuit, such as the electronics, i.e. circuitry, battery, and so forth can be placed in E.

FIG. 4B illustrates a disposable cartridge (200) where the air inlet (220) is aligned with the aerosol axis (223).

FIG. 5A illustrates a handheld medical device including a disposable cartridge (200) attached to a handheld controller (100). The disposable cartridge (200) includes an air inlet (220), and airway (203) and an air outlet configured as a mouthpiece (202). The handheld controller (100) includes the drug foil substrate heating circuit including a battery (101), and a microcontroller (102), and an air inlet controller extension (103). The air inlet controller extension (103) and the air inlet (220) are fluidly and tightly connected. The handheld controller (100) and the disposable cartridge (200) are connected via an electric (302) and, optionally, a data interface (301).

FIG. 5B is similar to FIG. 5A but without the air inlet controller extension (103) in the handheld controller (100). The air inlet (220) in the disposable cartridge (200), which may contain a baffle, is directly open to the atmosphere.

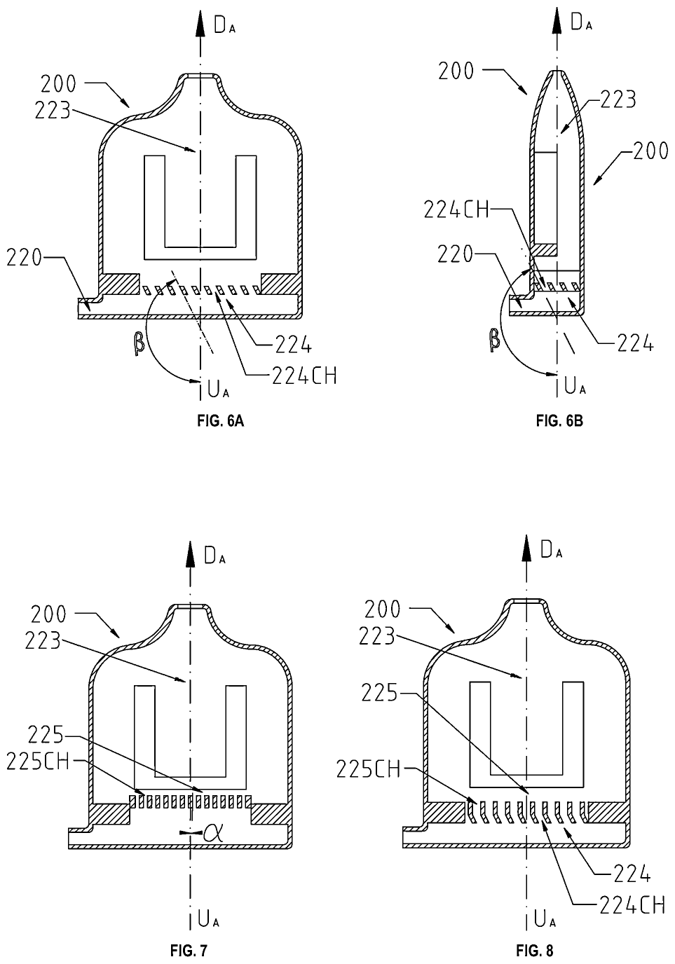

For simplicity, each element has been drawn in different figures (FIGS. 6A-12C), but all the possible combinations between these elements are deemed to be disclosed in the present document both in the form of a single handheld device and in the form of a disposable cartridge and a handheld controller.

FIG. 6A illustrates a schematic upper view of a disposable cartridge (200) with lateral air inlet (220), one airflow diverting structure (224) and the angle β defined by the intersection of the centerlines of the channels (224CH) of the airflow diverting structure (224) and the ray of the aerosol axis (223) in the upstream (U_{A}) direction measured in the sector containing the air inlet (220).

FIG. 6B illustrates a schematic side view of a disposable cartridge (200) with lower air inlet (220), one airflow diverting structure (224) and the angle β defined by the intersection of the centerlines of the channels (224CH) of the airflow diverting structure (224) and the ray of the aerosol axis (223) in the upstream (U_{A}) direction measured in the sector containing the air inlet (220).

FIG. 7 illustrates a schematic upper view of a disposable cartridge (200) showing one airflow straightening structure (225) parallel (α=) 0° to the aerosol axis (223). The angle α is defined by the intersection of the centerlines of the channels (225CH) and the ray of the aerosol axis (223) in the upstream (U_{A}) direction.

FIG. 8 illustrates a schematic upper view of a disposable cartridge (200) showing the channels (225CH) of the airflow straightening structure (225) attached to the channels (224CH) of the airflow diverting structure (224).

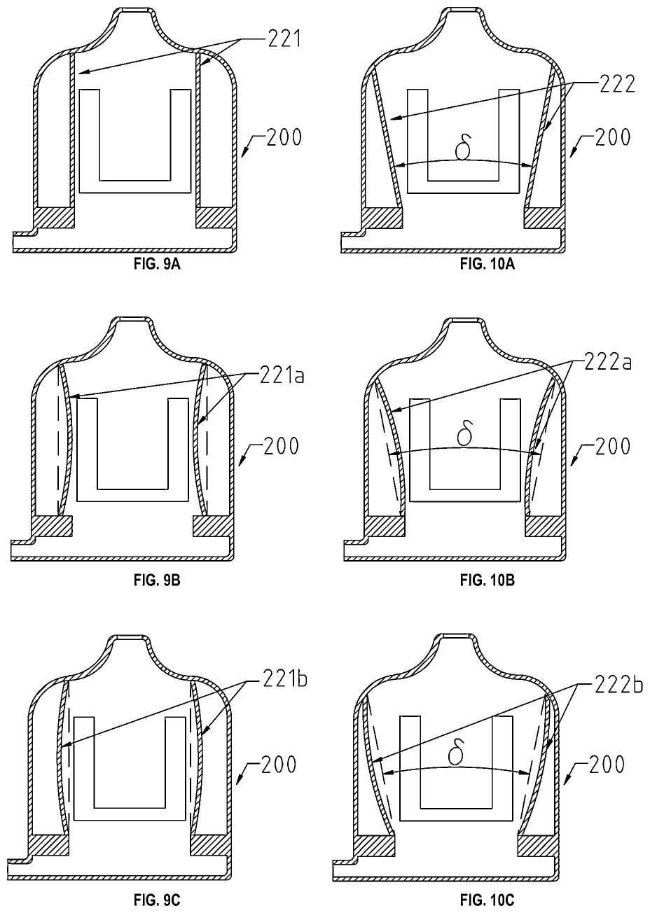

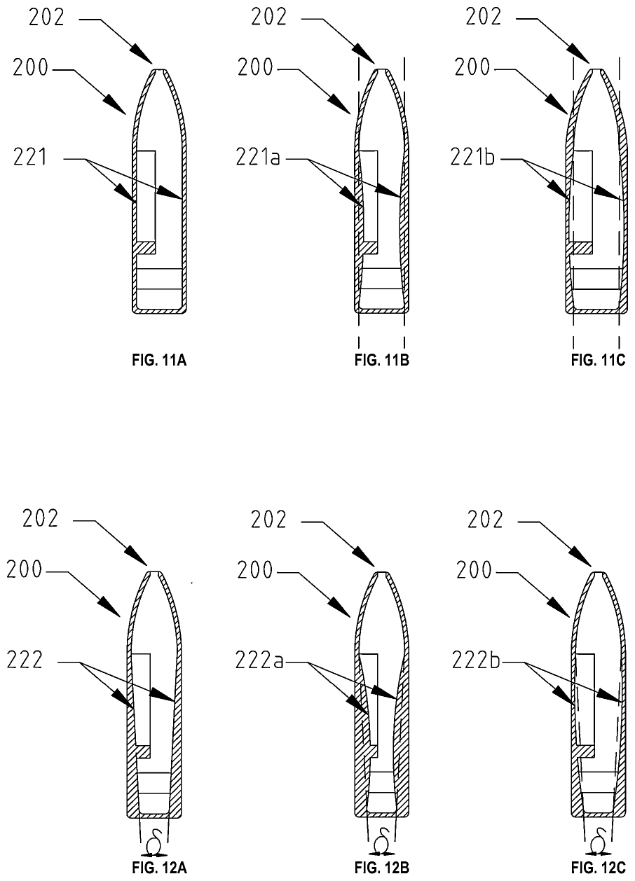

FIG. 9A-12C illustrate schematic views of a disposable cartridge (200). FIG. 9A-10C illustrate upper views of the disposable cartridge (200). FIGS. 11A-12C illustrate lateral views of the disposable cartridge (200). Parallel straight opposite walls (221) are shown in FIGS. 9A and 11A. Parallel convex opposite walls (221a) are shown in FIGS. 9B and 11B. Parallel concave opposite walls (221b) are shown in FIGS. 9C and 11C. Curved opposite walls (FIGS. 9B, 9C, 11B and 11C) are considered parallel when the chords (dashed lines) connecting the two curved extremes of the walls are parallel. Angled straight opposite walls (222) are shown in FIGS. 10A and 12A defining an angle δ. Angled convex opposite walls (222a) are shown in FIGS. 10B and 12B defining an angle δ. Angled concave opposite walls (222b) are shown in FIGS. 10C and 12C defining an angle δ. When the opposite walls are curved (FIGS. 10B, 10C, 12B and 12C) the angle δ is defined by the chords (dashed lines) of walls connecting the two curved extremes of the walls.

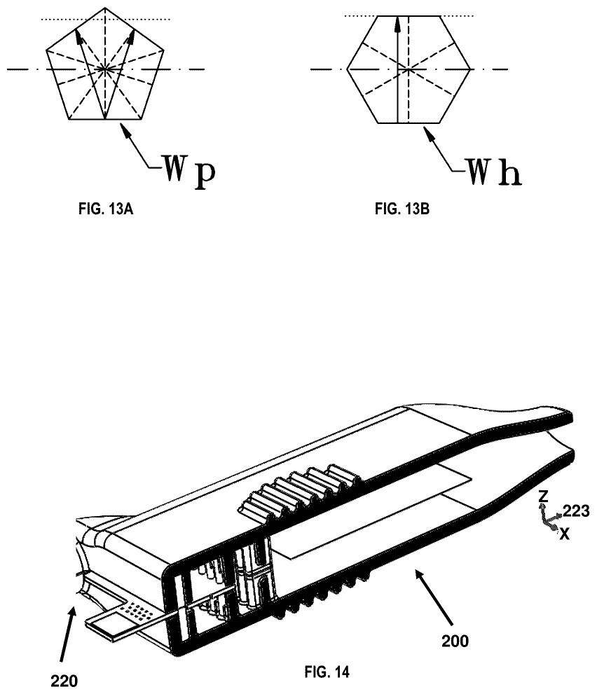

FIGS. 13A and 13B illustrate cross-sections of a handheld medical device with pentagonal (FIG. 13A) and hexagonal cross-sections (FIG. 13B) and show which walls are opposite.

In FIG. 13A the dashed lines represent the height of the pentagon and its crossing marks the center of the pentagon. The dash and dot line in FIG. 13A represents the plane that is parallel to the wall Wp and includes the geometrical center of the pentagonal cross-section of the handheld medical device. This plane is referred to as “the central plane”. The dotted line is the mirror image of the wall Wp with respect to the central plane. The walls crossed by the mirror image of the wall Wp with respect to the central plane (indicated with arrows) are the walls opposite to the wall Wp.

In FIG. 13B the dashed lines represent the minimal diameter of the hexagon and its crossing marks the center of the hexagon. The dash and dot line in FIG. 13B represents the plane that is parallel to the wall Wh and includes the geometrical center of the hexagonal cross-section of the handheld medical device. This plane is referred to as “the central plane”. The dotted line is the mirror image of the wall Wh with respect to the central plane. The wall parallel to the mirror image of the wall Wh with respect to the central plane (indicated with an arrow) is the wall opposite to the wall Wh.

If the walls of the device are curved the same methodology applies but using the chords of the walls.

FIG. 14 illustrates a cross-section of a disposable cartridge (200). The aerosol axis (223) corresponds to the cartridge length dimension. The plane defined by the aerosol axis (223) and the center of the air inlet (220) is referred to as the “aerosol axis-air inlet plane”. In FIG. 14 the aerosol axis-air inlet plane corresponds to the X-223 plane. Axis X, which is perpendicular to the aerosol axis (223), corresponds to the cartridge's lateral dimension.

In FIG. 14, the axis perpendicular to the aerosol axis-air inlet plane, Z-axis, corresponds to the height dimension. When the air inlet is in the vicinity of the aerosol axis, the Z-axis is perpendicular to the drug foil substrate plane. The air inlet is considered to be in the vicinity of the aerosol axis when it is within the 25% 75% of the lateral dimension of the cartridge or within the 25% 75% of the height of the cartridge.

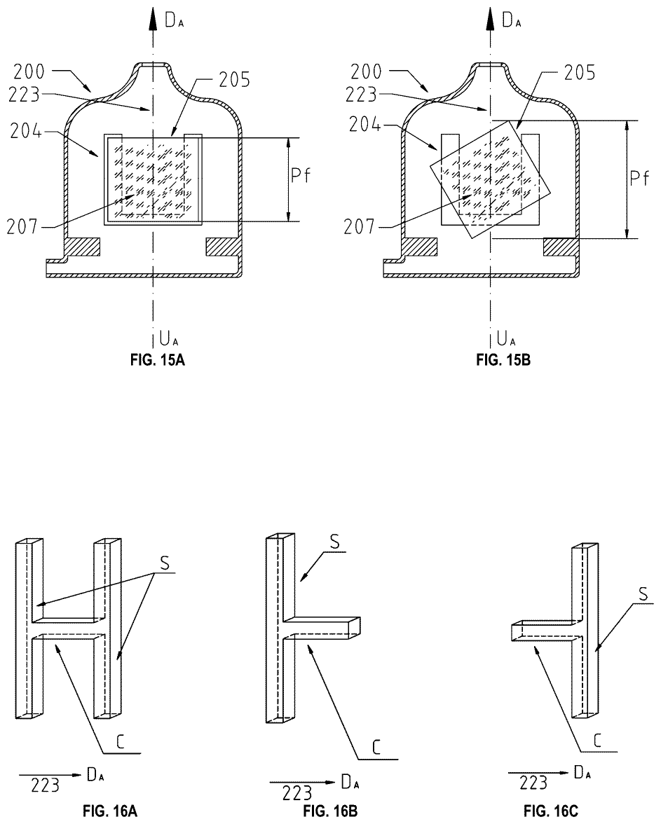

FIGS. 15A and 15B illustrate disposable cartridges (200) showing the drug foil substrate (205) placed in two different dispositions on a drug foil substrate support (204). The drug foil substrate (205) is coated with a solid drug film (207). Pf indicates the projection of the drug foil substrate (205) on the aerosol axis (223) of the two dispositions.

In some embodiments the one or more airflow diverting structures (224) and/or the one or more airflow straightening structures (225) may form cavities upstream and/or downstream the channels (224CH, 225CH). The channels (224CH, 225CH) and the cavities having the form of a three-dimensional (3D) H, right tack symbol (├) or left tack symbol (┤). In a more specific embodiment, FIG. 16A illustrates a 3D H, where the crossbar (C), which represents the channel (224CH, 225CH) of the one or more airflow diverting structures (224) or of the one or more airflow straightening structures (225) connecting the two stems(S), which each of the stems(S) represent the upstream and downstream cavities (224C, 225C). In FIG. 16A, the crossbar (C) is parallel to the aerosol axis (223). D_{A} indicates the downstream direction of the aerosol axis (223).

In another embodiment, FIG. 16B illustrates a 3D right tack symbol (├), where the crossbar (C), which represents the channel (224CH, 225CH) of the one or more airflow diverting structures (224) or of the one or more airflow straightening structures (225), is parallel to the aerosol axis (223). D_{A} indicates the downstream direction of the aerosol axis (223).

In another embodiment, FIG. 16C illustrates a 3D left tack symbol (┤), where the crossbar (C), which represents the channel (224CH, 225CH) of the one or more airflow diverting structures (224) or of the one or more airflow straightening structures (225), is parallel to the aerosol axis (223). D_{A} indicates the downstream direction of the aerosol axis (223).

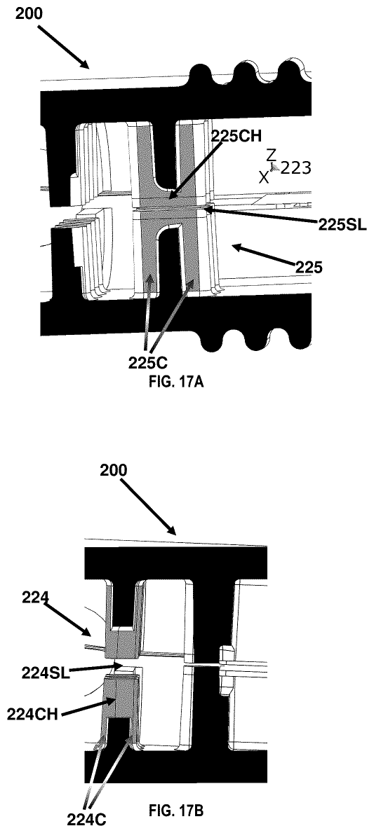

FIG. 17A illustrates a cross-section, parallel to the plane defined by the aerosol axis (223) and the Z-axis, of an embodiment of a disposable cartridge (200) showing the channel (225CH) and cavities (225C) of the airflow straightening structure (225) forming an “H” highlighted in grey. In this embodiment, there is a slit (225SL) parallel to the aerosol axis-air inlet plane connecting all the channels.

FIG. 17B illustrates a cross-section, parallel to plane defined by the centerline of a channel (224CH) of the airflow diverting structure (224) and the Z-axis, of an embodiment of a disposable cartridge (200) showing the channel (224CH) and cavities (224C) of the airflow diverting structure (224) forming an “H” highlighted in grey. In this embodiment, there is a slit (224SL) parallel to the aerosol axis-air inlet plane connecting all the channels.

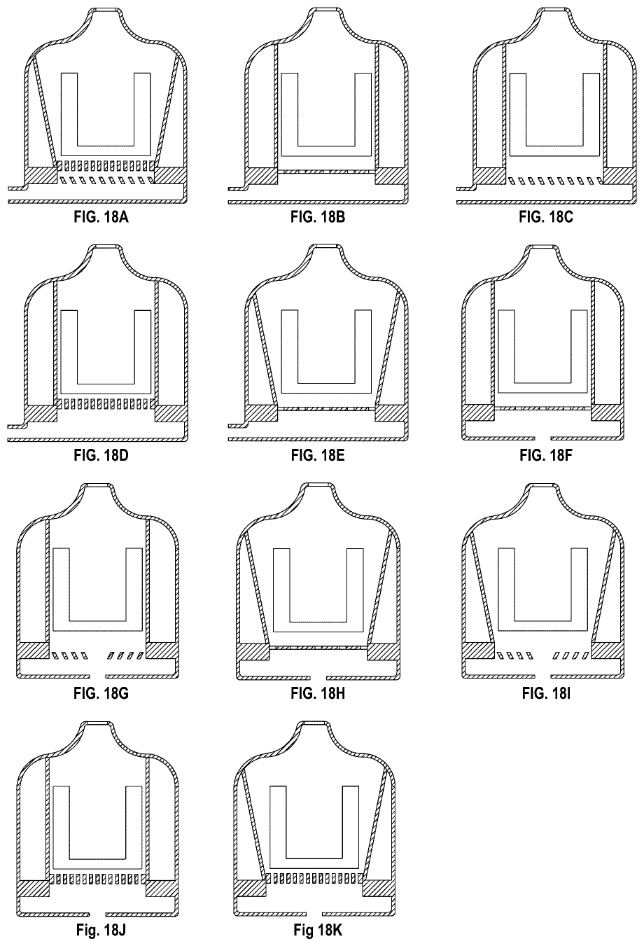

FIGS. 18A-18E illustrate devices C-G as described in the table Device Configurations in the Examples.

FIGS. 18F-18K illustrate devices H-M as disposable cartridges (200) with the air inlet (220) aligned with the aerosol axis (223) according to the following table:

| Device | H | I | J | K | L | M |
|---|---|---|---|---|---|---|
| Bulkhead | Yes | No | Yes | No | No | No |
| I. airflow diverting structure | No | No | No | No | No | No |
| airflow | | | | | | |
| II. airflow straightening | No | No | No | No | Yes | |
| structure | | | | | | |
| Ill. internal walls forming an | No | No | Yes | Yes | No | Yes |
| angle | | | | | | |
| IV. airflow directing features | No | Yes | No | Yes | No | No |
| Internal walls angle | 0° | 0° | 10° | 10° | 0° | 10° |
| Such as that in FIG. | 18F or 21F | 18G or 21G | 18H or 21H | 181 or 21I | 18J or 21J | 18K or 21K |

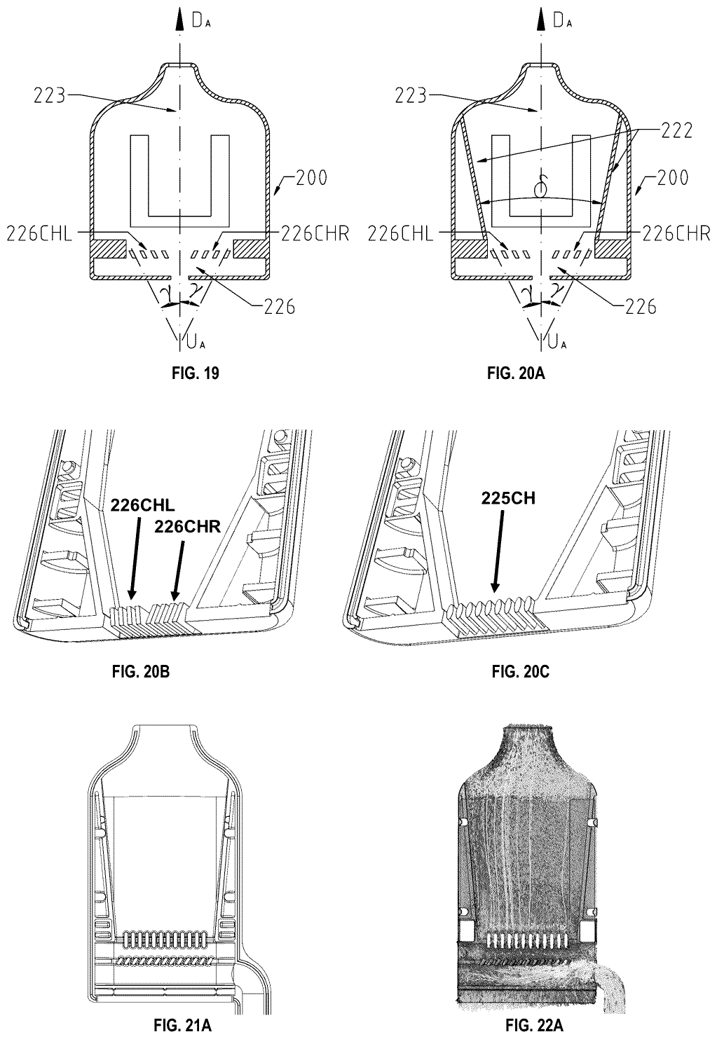

FIG. 19 illustrates a disposable cartridge (200) with airflow directing structures (226) including two sets of channels (226CHL and 226CHR). The angle γ formed by the channels with the aerosol axis (223) is as well indicated. The angle γ is measured upstream the drug foil substrate.

FIG. 20A illustrates a device with airflow directing structures (226) including two sets of channels (226CHL and 226CHR). The angle γ formed by the channels is as well indicated in combination with internal opposite walls (222) which define an angle δ. An actual embodiment of such device is illustrated in FIG. 211.

FIG. 20C illustrates airflow directing channels (226CHL and 226CHR) with rectangular shape.

FIG. 20D illustrates straightening channels (225CH) with aerodynamic elliptical shape.

Any of the diverting channels (224CH), straightening channels (225CH) and/or airflow directing channels (226CHL and 226CHR) optionally in combination with any of the Embodiment 1 to Embodiment 293 can have such shapes.

The handheld medical device may further include means for controlling the temperature of the drug foil substrate by sensing the temperature of the drug foil substrate and feeding the drug foil substrate temperature information to the drug foil substrate heating circuit to modify the electric current delivery in order to achieve the required temperature.

Said means for controlling the temperature are selected from measurement of electrical resistance across the drug foil substrate, optical measurement, and/or direct contact measurement with a thermocouple.

The cross section of a crossbar can have any shape, e.g. round, triangular, square, rectangle, etc. The corners can be square or round.

### EMBODIMENTS (AS NUMBERED BELOW)

Embodiment 1. A handheld medical device suitable to generate a drug condensation aerosol by thermal vaporization of a drug including:
   a. an airway (203) defined by internal walls;
   b. an air inlet (220) at one end of the airway (203);
   c. an air outlet (202), configured as a mouthpiece, at another end of the airway (203);
   d. a drug foil substrate (205) having an impermeable surface, with or without perforations, placed within the airway (203);
   e. a drug foil substrate support (204);
   f. a solid drug film (207) coated on the drug foil substrate (205);
   g. an aerosol axis (223) defined by the geometrical center of the solid drug film (207) and the geometrical center of the air outlet (202), where the downstream direction (D_{A}) goes from the geometrical center of the solid drug film to the geometrical center of the air outlet (202) and the upstream direction (U_{A}) is the opposite direction;
   h. a drug foil substrate heating circuit to vaporize at least part of the drug in the solid drug film (207) to produce condensation aerosol particles, where the drug foil substrate heating circuit includes at least one battery (101), and
   i. aerosol particle delivery modifiers selected from at least one of:
      I. one or more airflow diverting structures (224), which include a structure placed upstream of the drug foil substrate (205) including a plurality of channels (224CH), where the centerlines of the channels define an angle β from 100° to 170° with the ray of the aerosol axis (223) in the upstream (U_{A}) direction measured in the sector containing the air inlet (220);
      II. one or more airflow straightening structures (225), which include a structure (225) placed upstream the drug foil substrate (205) including a plurality of channels (225CH), where the centerlines of the channels define an angle α from −10° to 10° with the aerosol axis (223);
      III. internal walls including two internal opposite walls (222, 222a, 222b) which define an angle δ of 5° to 20° in at least 80% of its portion included within the projection (Pf) of the drug foil substrate (205) on the aerosol axis (223); where the vertex of said angle δ is located in the upstream (U_{A}) direction; and
      IV. one or more airflow directing structures (226), which include a structure (226) placed upstream the drug foil substrate (205) including at least two sets of channels (226CHL and 226CHR), one set at each side of the Z-plane, where the centerlines of each set of channels (226CHL and 226CHR) define an angle γ from 5° to 20° with the Z-plane; where the vertex of said angle γ is located upstream the drug foil substrate.
Embodiment 2. The device of the previous Embodiment, where the drug foil substrate heating circuit includes an electrical heater.
Embodiment 3. The device of the previous Embodiment, which includes a disposable cartridge (200) and a handheld controller (100), where the disposable cartridge (200) is suitable to be connected to the handheld controller (100), the disposable cartridge (200) includes features a) to g), and i) according to Embodiment 1, and the handheld controller (100) includes feature h) according to Embodiment 1.
Embodiment 4. The device of the previous Embodiment, where the air inlet (220) of the disposable cartridge (200) is fluidly connected to an air inlet controller extension (103) of the handheld controller (100).
Embodiment 5. The device of the previous Embodiment, where the air inlet (220) of the disposable cartridge (200) is connected to the air inlet controller extension (103) of the handheld controller (100) through a pneumatic sealing.
Embodiment 6. The device of Embodiment 3, where the air inlet (220) of the disposable cartridge (200) is not fluidly connected to the handheld controller (100).
Embodiment 7. A disposable cartridge (200) suitable to be connected to a handheld controller (100) which includes features a) to g), and i) according to Embodiment 1.
Embodiment 8. The disposable cartridge (200) of Embodiment 7, where the air inlet (220) includes a pneumatic sealing.
Embodiment 9. The disposable cartridge (200) of any of the Embodiment 7 to Embodiment 8, which externally is 60-160 mm×50-85 mm×9-35 mm
Embodiment 10. The disposable cartridge (200) of any of the Embodiment 7 to Embodiment 9, which internally is 60-160 mm×20-70 mm×5-25 mm.
Embodiment 11. The disposable cartridge (200) of any of the Embodiment 7 to Embodiment 10, which externally is 85-135 mm×50-75 mm×9-30 mm.
Embodiment 12. The disposable cartridge (200) of any of the Embodiment 7 to Embodiment 11, which internally is 80-135 mm×25-50 mm×10-22 mm.
Embodiment 13. The disposable cartridge (200) of any of the Embodiment 7 to Embodiment 11, which internally is 80-135 mm×25-50 mm×5-15 mm.
Embodiment 14. The device or disposable cartridge (200) of any of the preceding Embodiments, which further includes.
   a. at least one microcontroller (102);
   b. an airflow detector which triggers the heating of the drug foil substrate (205); and
   c. electrical (302) and/or data connections (301) between the at least one battery (101), the at least one microcontroller (102), and the drug foil substrate (205).
Embodiment 15. The device or disposable cartridge (200) of any of the preceding Embodiments, where the aerosol particle delivery modifiers includes feature I according to Embodiment 1.
Embodiment 16. The device or disposable cartridge (200) of any of the preceding Embodiments, where the aerosol particle delivery modifiers includes feature II according to Embodiment 1.
Embodiment 17. The device or disposable cartridge (200) of any of the preceding Embodiments, where the aerosol particle delivery modifiers includes feature III according to Embodiment 1.
Embodiment 18. The device or disposable cartridge (200) of any of the preceding Embodiments, where the aerosol particle delivery modifiers includes feature IV according to Embodiment 1.
Embodiment 19. The device or disposable cartridge (200) of any of the preceding Embodiments, where the aerosol particle delivery modifiers includes features I and II according to Embodiment 1.
Embodiment 20. The device or disposable cartridge (200) of any of the preceding Embodiments, where the aerosol particle delivery modifiers includes features I and III according to Embodiment 1.
Embodiment 21. The device or disposable cartridge (200) of any of the preceding Embodiments, where the aerosol particle delivery modifiers includes features I and IV according to Embodiment 1.
Embodiment 22. The device or disposable cartridge (200) of any of the preceding Embodiments, where the aerosol particle delivery modifiers includes features II and III according to Embodiment 1.
Embodiment 23. The device or disposable cartridge (200) of any of the preceding Embodiments, where the aerosol particle delivery modifiers includes features II and IV according to Embodiment 1.
Embodiment 24. The device or disposable cartridge (200) of any of the preceding Embodiments, where the aerosol particle delivery modifiers includes features III and IV according to Embodiment 1.
Embodiment 25. The device or disposable cartridge (200) of any of the preceding Embodiments, where the aerosol particle delivery modifiers includes features I, II, and III according to Embodiment 1.
Embodiment 26. The device or disposable cartridge (200) of any of the preceding Embodiments, where the aerosol particle delivery modifiers includes features I, II, and IV according to Embodiment 1.
Embodiment 27. The device or disposable cartridge (200) of any of the preceding Embodiments, where the aerosol particle delivery modifiers includes features I, III, and IV according to Embodiment 1.
Embodiment 28. The device or disposable cartridge (200) of any of the preceding Embodiments, where the aerosol particle delivery modifiers includes features II, III, and IV according to Embodiment 1.
Embodiment 29. The device or disposable cartridge (200) of any of the preceding Embodiments, where the aerosol particle delivery modifiers includes features I, II, III, and IV according to Embodiment 1.
Embodiment 30. The device or disposable cartridge (200) of any of previous Embodiments, where the aerosol axis (223) crosses within the central region that includes half of the channels of the most upstream of the one or more airflow diverting structures (224) and the one or more airflow straightening structures (225).
Embodiment 31. The device or disposable cartridge (200) of the preceding Embodiment, where the aerosol axis (223) crosses within the central region that includes a third of the channels of the most upstream of the one or more airflow diverting structures (224) and the one or more airflow straightening structures (225).
Embodiment 32. The device or disposable cartridge (200) of the preceding Embodiment, where the aerosol axis (223) crosses within the central region that includes a quarter of the channels of the most upstream of the one or more airflow diverting structures (224) and the one or more airflow straightening structures (225).
Embodiment 33. The device or disposable cartridge (200) of any of the Embodiments 1 to Embodiment 14, Embodiment 18 to Embodiment 20, Embodiment 24 to Embodiment 26 or Embodiment 28 to Embodiment 31, where the angle β ranges from 105° to 160°.
Embodiment 34. The device or disposable cartridge (200) of the preceding Embodiment, where the angle β ranges from 110° to 150°.
Embodiment 35. The device or disposable cartridge (200) of the preceding Embodiment, where the angle β ranges from 115° to 140°.
Embodiment 36. The device or disposable cartridge (200) of the preceding Embodiment, where the angle β ranges from 120° to 130°.
Embodiment 37. The device or disposable cartridge (200) of any of the Embodiments 1 to Embodiment 14, Embodiment 18 to Embodiment 20, Embodiment 24 to Embodiment 26 or Embodiment 28 to Embodiment 35, where the channels (224CH) of the one or more airflow diverting structures (224) are curved and the angle β is measured using the axis defined by the center of the upstream mouth of the channel (224CH) and the center of the downstream mouth of the channel (224CH) instead of the centerline.
Embodiment 38. The device or disposable cartridge (200) of any of the Embodiments 1 to Embodiment 14, Embodiment 18 to Embodiment 20, Embodiment 24 to Embodiment 26 or Embodiment 28 to Embodiment 36, where the channels (224CH) of the one or more airflow diverting structures (224) extend from 0.7 to 5 mm measured in the centerline direction.
Embodiment 39. The device or disposable cartridge (200) of the preceding Embodiment, where the channels (224CH) of the one or more airflow diverting structures (224) extend from 0.8 to 4.5 mm.
Embodiment 40. The device or disposable cartridge (200) of the preceding Embodiment, where the channels (224CH) of the one or more airflow diverting structures (224) extend from 0.9 to 4 mm.
Embodiment 41. The device or disposable cartridge (200) of the preceding Embodiment, where the channels (224CH) of the one or more airflow diverting structures (224) extend from 1 to 3.5 mm.
Embodiment 42. The device or disposable cartridge (200) of the preceding Embodiment, where the channels (224CH) of the one or more airflow diverting structures (224) extend from 1.1 to 3 mm.
Embodiment 43. The device or disposable cartridge (200) of the preceding Embodiment, where the channels (224CH) of the one or more airflow diverting structures (224) extend from 1.2 to 2.5 mm.
Embodiment 44. The device or disposable cartridge (200) of the preceding Embodiment, where the channels (224CH) of the one or more airflow diverting structures (224) extend from 1.3 to 2.0 mm.
Embodiment 45. The device or disposable cartridge (200) of any of the Embodiments 1 to Embodiment 14, Embodiment 18 to Embodiment 20, Embodiment 24 to Embodiment 26 or Embodiment 28 to Embodiment 43, where minimum width of the channels (224CH) of the one or more airflow diverting structures (224) ranges from 0.4 to 2 mm.
Embodiment 46. The device or disposable cartridge (200) of the preceding Embodiment, where the minimum width of the channels (224CH) of the one or more airflow diverting structures (224) ranges from 0.5 to 1.5 mm.
Embodiment 47. The device or disposable cartridge (200) of the preceding Embodiment, where the minimum width of the channels (224CH) of the one or more airflow diverting structures (224) ranges from 0.6 to 1.4 mm.
Embodiment 48. The device or disposable cartridge (200) of the preceding Embodiment, where the minimum width of the channels (224CH) of the one or more airflow diverting structures (224) ranges from 0.7 to 1.3 mm.
Embodiment 49. The device or disposable cartridge (200) of the preceding Embodiment, where the minimum width of the channels (224CH) of the one or more airflow diverting structures (224) ranges from 0.75 to 1.2 mm.
Embodiment 50. The device or disposable cartridge (200) of the Embodiments 1 to Embodiment 14, Embodiment 18 to Embodiment 20, Embodiment 24 to Embodiment 26 or Embodiment 28 to Embodiment 48, where the minimum height of the channels (224CH) of the one or more airflow diverting structures (224) ranges from 3 to 15 mm.
Embodiment 51. The device or disposable cartridge (200) of the preceding Embodiment, where the minimum height of the channels (224CH) of the one or more airflow diverting structures (224) ranges from 3.5 to 12 mm.
Embodiment 52. The device or disposable cartridge (200) of the preceding Embodiment, where the minimum height of the channels (224CH) of the one or more airflow diverting structures (224) ranges from 4 to 10 mm.
Embodiment 53. The device or disposable cartridge (200) of the preceding Embodiment, where the minimum height of the channels (224CH) of the one or more airflow diverting structures (224) ranges from 4.5 to 8 mm.
Embodiment 54. The device or disposable cartridge (200) of the preceding Embodiment, where the minimum height of the channels (224CH) of the one or more airflow diverting structures (224) ranges from 5 to 7.5 mm.
Embodiment 55. The device or disposable cartridge (200) of the Embodiments 1 to Embodiment 14, Embodiment 18 to Embodiment 20, Embodiment 24 to Embodiment 26 or Embodiment 28 to Embodiment 53, where the minimum width to height ratio of the channels (224CH) of the one or more airflow diverting structures (224) ranges from 1:1.1 to 1:40.
Embodiment 56. The device or disposable cartridge (200) of the preceding Embodiment, where the minimum width to height ratio of the channels (224CH) of the one or more airflow diverting structures (224) ranges from 1:2 to 1:30.
Embodiment 57. The device or disposable cartridge (200) of the preceding Embodiment, where the minimum width to height ratio of the channels (224CH) of the one or more airflow diverting structures (224) ranges from 1:3 to 1:30.
Embodiment 58. The device or disposable cartridge (200) of the preceding Embodiment, where the minimum width to height ratio of the channels (224CH) of the one or more airflow diverting structures (224) ranges from 1:4 to 1:25.
Embodiment 59. The device or disposable cartridge (200) of the preceding Embodiment, where the minimum width to height ratio of the channels (224CH) of the one or more airflow diverting structures (224) ranges from 1:5 to 1:20.
Embodiment 60. The device or disposable cartridge (200) of the preceding Embodiment, where the minimum width to height ratio of the channels (224CH) of the one or more airflow diverting structures (224) ranges from 1:6 to 1:15.
Embodiment 61. The device or disposable cartridge (200) of the preceding Embodiment, where the minimum width to height ratio of the channels (224CH) of the one or more airflow diverting structures (224) ranges from 1:6.5 to 1:10.
Embodiment 62. The device or disposable cartridge (200) of any of the Embodiments 1 to Embodiment 14, Embodiment 18 to Embodiment 20, Embodiment 24 to Embodiment 26 or Embodiment 28 to Embodiment 60, where the centerlines of any two closer channels (224CH) of the one or more airflow diverting structures (224) are from 1 to 5 mm apart from each other measured from centerline to centerline at the downstream mouth of the channels (224CH).
Embodiment 63. The device or disposable cartridge (200) of the preceding Embodiment, where the centerlines of any two closer channels (224CH) of the one or more airflow diverting structures (224) are from 1.25 to 4.5 mm apart.
Embodiment 64. The device or disposable cartridge (200) of the preceding Embodiment, where the centerlines of any two closer channels (224CH) of the one or more airflow diverting structures (224) are from 1.5 to 4.0 mm apart.
Embodiment 65. The device or disposable cartridge (200) of the preceding Embodiment, where the centerlines of any two closer channels (224CH) of the one or more airflow diverting structures (224) are from 1.75 to 3.75 mm apart.
Embodiment 66. The device or disposable cartridge (200) of the preceding Embodiment, where the centerlines of any two closer channels (224CH) of the one or more airflow diverting structures (224) are from 2 to 3 mm apart.
Embodiment 67. The device or disposable cartridge (200) of any of the Embodiments 1 to Embodiment 14, Embodiment 18 to Embodiment 20, Embodiment 24 to Embodiment 26 or Embodiment 28 to Embodiment 65, where the one or more airflow diverting structures (224) include between 5 and 20 channels (224CH).
Embodiment 68. The device or disposable cartridge (200) of the preceding Embodiment, where the one or more airflow diverting structures (224) include between 8 and 17 channels (224CH).
Embodiment 69. The device or disposable cartridge (200) of the preceding Embodiment, where the one or more airflow diverting structures (224) include between 10 and 15 channels (224CH).
Embodiment 70. The device or disposable cartridge (200) of the preceding Embodiment, where the one or more airflow diverting structures (224) include between 11 and 14 channels (224CH).
Embodiment 71. The device or disposable cartridge (200) of any of the Embodiments 1 to Embodiment 14, Embodiment 18 to Embodiment 20, Embodiment 24 to Embodiment 26 or Embodiment 28 to Embodiment 69, where the centerlines of the channels (224CH) of the one or more airflow diverting structures (224) are within 25% to 75% of the internal height of the handheld medical device measured immediately downstream of the one or more airflow diverting structures (224).
Embodiment 72. The device or disposable cartridge (200) of the preceding Embodiment, where the centerlines of the channels (224CH) of the one or more airflow diverting structures (224) are within 33% to 66% of the internal height of the handheld medical device measured immediately downstream of the one or more airflow diverting structures (224).
Embodiment 73. The device or disposable cartridge (200) of the preceding Embodiment, where the centerlines of the channels (224CH) of the one or more airflow diverting structures (224) are within 37% to 63% of the internal height of the handheld medical device measured immediately downstream of the one or more airflow diverting structures (224).
Embodiment 74. The device or disposable cartridge (200) of any of the Embodiments 1 to Embodiment 14, Embodiment 18 to Embodiment 20, Embodiment 24 to Embodiment 26 or Embodiment 28 to Embodiment 72, where the centerlines of the channels (224CH) of the one or more airflow diverting structures (224) are within 5% to 33% and/or 95% to 66% of the internal height of the handheld medical device measured immediately downstream of the one or more airflow diverting structures (224).
Embodiment 75. The device or disposable cartridge (200) of the preceding Embodiment, where the centerlines of the channels (224CH) of the one or more airflow diverting structures (224) are within 10% to 25% and/or 90 to 75% of the internal height of the handheld medical device measured immediately downstream of the one or more airflow diverting structures (224).
Embodiment 76. The device or disposable cartridge (200) of the preceding Embodiment, where the centerlines of the channels (224CH) of the one or more airflow diverting structures (224) are within 10% to 20% and/or 90% to 80% of the internal height of the handheld medical device measured immediately downstream of the one or more airflow diverting structures (224).
Embodiment 77. The device or disposable cartridge (200) of the preceding Embodiment, where the centerlines of the channels (224CH) of the one or more airflow diverting structures (224) are within 10% to 15% and/or 90% to 85% of the internal height of the handheld medical device measured immediately downstream of the one or more airflow diverting structures (224).
Embodiment 78. The device or disposable cartridge (200) of any of the Embodiments 1 to Embodiment 14, Embodiment 18 to Embodiment 20, Embodiment 24 to Embodiment 26 or Embodiment 28 to Embodiment 76, where at least part of the channels (224CH) of the one or more airflow diverting structures (224) are connected by a slit (224SL).
Embodiment 79. The device or disposable cartridge (200) of the preceding Embodiment, where the central plane of the slit (224SL) forms a −10° to 10° angle with the aerosol axis-air inlet plane, which is the plane defined by the aerosol axis (223) and the center of the air inlet (220).
Embodiment 80. The device or disposable cartridge (200) of the preceding Embodiment, where the central plane of the slit (224SL) forms a −5° to 5° angle with the aerosol axis-air inlet plane.
Embodiment 81. The device or disposable cartridge (200) of the preceding Embodiment, where the central plane of the slit (224SL) forms a −2° to 2° angle with the aerosol axis-air inlet plane.
Embodiment 82. The device or disposable cartridge (200) of the Embodiments 1 to Embodiment 14, Embodiment 18 to Embodiment 20, Embodiment 24 to Embodiment 26 or Embodiment 28 to Embodiment 80, where the slit (224SL) ranges from 0.1 to 3 mm high.
Embodiment 83. The device or disposable cartridge (200) of the preceding Embodiment, where the slit (224SL) ranges from 0.25 to 2.5 mm high.
Embodiment 84. The device or disposable cartridge (200) of the preceding Embodiment, where the slit (224SL) ranges from 0.5 to 2.25 mm high.
Embodiment 85. The device or disposable cartridge (200) of the preceding Embodiment, where the slit (224SL) ranges from 0.75 to 2 mm high.
Embodiment 86. The device or disposable cartridge (200) of the preceding Embodiment, where the slit (224SL) ranges from 1 to 2 mm high.
Embodiment 87. The device or disposable cartridge (200) of the preceding Embodiment, where the slit (224SL) ranges from 1.25 to 1.75 mm high.
Embodiment 88. The device or disposable cartridge (200) of any of the Embodiments 1 to Embodiment 14, Embodiment 18 to Embodiment 20, Embodiment 24 to Embodiment 26 or Embodiment 28 to Embodiment 86, where both most lateral channels (224CH) of the one or more airflow diverting structures (224) are up to four times wider than the rest of the channels (224CH) of the one or more airflow diverting structures (224).
Embodiment 89. The device or disposable cartridge (200) of any of the Embodiments 1 to Embodiment 14, Embodiment 18 to Embodiment 20, Embodiment 24 to Embodiment 26 or Embodiment 28 to Embodiment 87, where the one or more airflow diverting structures (224) include cavities (224C) upstream and/or downstream the channels (224CH).
Embodiment 90. The device or disposable cartridge (200) of the preceding Embodiment, where the channels (224CH) and the cavities (224C) are
   a. H shaped,
   b. right tack shaped (├), or
   c. left tack shaped (┤); and
      the channels (224CH) being the crossbar (C) of the H, ├ or ┤; the cavities (224C) being the stems(S) of the H, ├ or ┤; and the cavities (224C) extending up to 100% of the internal height of the device the closest of just downstream or upstream of the one or more airflow diverting structures (224).
Embodiment 91. The device or disposable cartridge (200) of the preceding Embodiment, where the crossbar-stems plane forms an angle from 80° to 100° with the aerosol axis-air inlet plane, which is the plane defined by the aerosol axis (223) and the center of the air inlet (220).
Embodiment 92. The device or disposable cartridge (200) of the preceding Embodiment, where the crossbar-stems plane forms an angle from 85° to 95° with the aerosol axis-air inlet plane.
Embodiment 93. The device or disposable cartridge (200) of any of the Embodiment 89 to Embodiment 91, where the cavities (224C) extend from 0.1 to 2.5 mm upstream (U_{A}) and/or downstream (D_{A}) the channels (224CH) of the one or more airflow diverting structures (224) measured in the centerline direction.
Embodiment 94. The device or disposable cartridge (200) of the preceding Embodiment, where the cavities (224C) extend from 0.15 to 2 mm upstream (U_{A}) and/or downstream (D_{A}) the channels (224CH) of the one or more airflow diverting structures (224) measured in the centerline direction.
Embodiment 95. The device or disposable cartridge (200) of the preceding Embodiment, where the cavities (224C) extend from 0.2 to 1.5 mm upstream (U_{A}) and/or downstream (D_{A}) the channels (224CH) of the one or more airflow diverting structures (224) measured in the centerline direction.
Embodiment 96. The device or disposable cartridge (200) of the preceding Embodiment, where the cavities (224C) extend from 0.25 to 1 mm upstream (U_{A}) and/or downstream (D_{A}) the channels (224CH) of the one or more airflow diverting structures (224) measured in the centerline direction.
Embodiment 97. The device or disposable cartridge (200) of the preceding Embodiment, where the cavities (224C) extend from 0.3 to 0.75 mm upstream (U_{A}) and/or downstream (D_{A}) the channels (224CH) of the one or more airflow diverting structures (224) measured in the centerline direction.
Embodiment 98. The device or disposable cartridge (200) of any of the Embodiment 89 to Embodiment 96, where the width of the cavities (224C), in its narrowest section, are 100 to 125% as width as the channels (224CH) of the one or more airflow diverting structures (224).
Embodiment 99. The device or disposable cartridge (200) of any of the Embodiments 1 to Embodiment 13, Embodiment 15, Embodiment 18, Embodiment 21 to Embodiment 22, Embodiment 24 to Embodiment 25 or Embodiment 27 to Embodiment 97, where angle α ranges from −5 to 5°.
Embodiment 100. The device or disposable cartridge (200) of the preceding Embodiment, where the angle α ranges from −2 to 2°.
Embodiment 101. The device or disposable cartridge (200) of any of the Embodiments 1 to Embodiment 13, Embodiment 15, Embodiment 18, Embodiment 21, Embodiment 22, Embodiment 24, Embodiment 25 or Embodiment 27 to Embodiment 99, where the channels (225CH) of the one or more airflow straightening structures (225) are curved and the angle α is measured using the axis defined by the center of the upstream mouth of the channel (225CH) and the center of the downstream mouth of the channel (225CH) instead of the centerline.
Embodiment 102. The device or disposable cartridge (200) of any of the Embodiments 1 to Embodiment 13, Embodiment 15, Embodiment 18, Embodiment 21, Embodiment 22, Embodiment 24, Embodiment 25 or Embodiment 27 to Embodiment 100, where the channels (225CH) of the one or more airflow straightening structures (225) extend from 0.9 to 20 mm long measured in the centerline direction.
Embodiment 103. The device or disposable cartridge (200) of the preceding Embodiment, where the channels (225CH) of the one or more airflow straightening structures (225) extend from 0.9 to 15 mm.
Embodiment 104. The device or disposable cartridge (200) of the preceding Embodiment, where the channels (225CH) of the one or more airflow straightening structures (225) extend from 0.9 to 12 mm.
Embodiment 105. The device or disposable cartridge (200) of the preceding Embodiment, where the channels (225CH) of the one or more airflow straightening structures (225) extend from 0.9 to 13 mm.
Embodiment 106. The device or disposable cartridge (200) of the preceding Embodiment, where the channels (225CH) of the one or more airflow straightening structures (225) extend from 0.9 to 10 mm.
Embodiment 107. The device or disposable cartridge (200) of the preceding Embodiment, where the channels (225CH) of the one or more airflow straightening structures (225) extend from 0.9 to 7 mm.
Embodiment 108. The device or disposable cartridge (200) of the preceding Embodiment, where the channels (225CH) of the one or more airflow straightening structures (225) extend from 1 to 5 mm.
Embodiment 109. The device or disposable cartridge (200) of the preceding Embodiment, where the channels (225CH) of the one or more airflow straightening structures (225) extend from 1 to 4 mm.
Embodiment 110. The device or disposable cartridge (200) of the preceding Embodiment, where the channels (225CH) of the one or more airflow straightening structures (225) extend from 1 to 3 mm.
Embodiment 111. The device or disposable cartridge (200) of the preceding Embodiment, where the channels (225CH) of the one or more airflow straightening structures (225) extend from 1 to 2 mm.
Embodiment 112. The device or disposable cartridge (200) of any of the Embodiments 1 to Embodiment 13, Embodiment 15, Embodiment 18, Embodiment 21, Embodiment 22, Embodiment 24, Embodiment 25 or Embodiment 27 to Embodiment 110, where the minimum width of channels (225CH) of the one or more airflow straightening structures (225) ranges from 0.4 to 4 mm.
Embodiment 113. The device or disposable cartridge (200) of the preceding Embodiment, where minimum width of the channels (225CH) of the one or more airflow straightening structures (225) ranges from 0.4 to 3 mm.
Embodiment 114. The device or disposable cartridge (200) of the preceding Embodiment, where minimum width of the channels (225CH) of the one or more airflow straightening structures (225) ranges from 0.4 to 2 mm.
Embodiment 115. The device or disposable cartridge (200) of the preceding Embodiment, where minimum width of the channels (225CH) of the one or more airflow straightening structures (225) ranges from 0.4 to 1.5 mm.
Embodiment 116. The device or disposable cartridge (200) of the preceding Embodiment, where minimum width of the channels (225CH) of the one or more airflow straightening structures (225) ranges from 0.5 to 1.5 mm.
Embodiment 117. The device or disposable cartridge (200) of the preceding Embodiment, where minimum width of the channels (225CH) of the one or more airflow straightening structures (225) ranges from 0.6 to 1.5 mm.
Embodiment 118. The device or disposable cartridge (200) of the preceding Embodiment, where minimum width of the channels (225CH) of the one or more airflow straightening structures (225) ranges from 0.6 to 1.4 mm.
Embodiment 119. The device or disposable cartridge (200) of the preceding Embodiment, where minimum width of the channels (225CH) of the one or more airflow straightening structures (225) ranges from 0.7 to 1.4 mm.
Embodiment 120. The device or disposable cartridge (200) of the preceding Embodiment, where minimum width of the channels (225CH) of the one or more airflow straightening structures (225) ranges from 0.8 to 1.4 mm.
Embodiment 121. The device or disposable cartridge (200) of the preceding Embodiment, where minimum width of the channels (225CH) of the one or more airflow straightening structures (225) ranges from 0.9 to 1.3 mm.
Embodiment 122. The device or disposable cartridge (200) of the Embodiments 1 to Embodiment 13, Embodiment 15, Embodiment 18, Embodiment 21, Embodiment 22, Embodiment 24, Embodiment 25 or Embodiment 27 to Embodiment 120, where minimum height of the channels (225CH) of the one or more airflow straightening structures (225) ranges from 1 to 15 mm.
Embodiment 123. The device or disposable cartridge (200) of the preceding Embodiment, where minimum height of the channels (225CH) of the one or more airflow straightening structures (225) ranges from 1.1 to 14 mm.
Embodiment 124. The device or disposable cartridge (200) of the preceding Embodiment, where minimum height of the channels (225CH) of the one or more airflow straightening structures (225) ranges from 1.2 to 13 mm.
Embodiment 125. The device or disposable cartridge (200) of the preceding Embodiment, where minimum height of the channels (225CH) of the one or more airflow straightening structures (225) ranges from 1.3 to 12 mm.
Embodiment 126. The device or disposable cartridge (200) of the preceding Embodiment, where minimum height of the channels (225CH) of the one or more airflow straightening structures (225) ranges from 1.4 to 11 mm.
Embodiment 127. The device or disposable cartridge (200) of the preceding Embodiment, where minimum height of the channels (225CH) of the one or more airflow straightening structures (225) ranges from 1.5 to 10 mm.
Embodiment 128. The device or disposable cartridge (200) of the preceding Embodiment, where minimum height of the channels (225CH) of the one or more airflow straightening structures (225) ranges from 1.6 to 8 mm.
Embodiment 129. The device or disposable cartridge (200) of the preceding Embodiment, where minimum height of the channels (225CH) of the one or more airflow straightening structures (225) ranges from 1.6 to 6 mm.
Embodiment 130. The device or disposable cartridge (200) of the preceding Embodiment, where minimum height of the channels (225CH) of the one or more airflow straightening structures (225) ranges from 1.6 to 5 mm.
Embodiment 131. The device or disposable cartridge (200) of the preceding Embodiment, where minimum height of the channels (225CH) of the one or more airflow straightening structures (225) ranges from 1.7 to 4 mm.
Embodiment 132. The device or disposable cartridge (200) of the preceding Embodiment, where minimum height of the channels (225CH) of the one or more airflow straightening structures (225) ranges from 1.8 to 3.5 mm.
Embodiment 133. The device or disposable cartridge (200) of the preceding Embodiment, where minimum height of the channels (225CH) of the one or more airflow straightening structures (225) ranges from 2 to 3 mm.
Embodiment 134. The device or disposable cartridge (200) of the Embodiments 1 to Embodiment 13, Embodiment 15, Embodiment 18, Embodiment 21, Embodiment 22, Embodiment 24, Embodiment 25 or Embodiment 27 to Embodiment 132, where the minimum width to height ratio of the channels (225CH) of the one or more airflow straightening structures (225) ranges from 1:1.1 to 1:30.
Embodiment 135. The device or disposable cartridge (200) of the preceding Embodiment, where the minimum width to height ratio of the channels (225CH) of the one or more airflow straightening structures (225) ranges from 1:1.2 to 1:20.
Embodiment 136. The device or disposable cartridge (200) of the preceding Embodiment, where the minimum width to height ratio of the channels (225CH) of the one or more airflow straightening structures (225) ranges from 1:1.2 to 1:15.
Embodiment 137. The device or disposable cartridge (200) of the preceding Embodiment, where the minimum width to height ratio of the channels (225CH) of the one or more airflow straightening structures (225) ranges from 1:1.2 to 1:10.
Embodiment 138. The device or disposable cartridge (200) of the preceding Embodiment, where the minimum width to height ratio of the channels (225CH) of the one or more airflow straightening structures (225) ranges from 1:1.2 to 1:9.
Embodiment 139. The device or disposable cartridge (200) of the preceding Embodiment, where the minimum width to height ratio of the channels (225CH) of the one or more airflow straightening structures (225) ranges from 1:1.2 to 1:8.
Embodiment 140. The device or disposable cartridge (200) of the preceding Embodiment, where the minimum width to height ratio of the channels (225CH) of the one or more airflow straightening structures (225) ranges from 1:1.4 to 1:6.
Embodiment 141. The device or disposable cartridge (200) of the preceding Embodiment, where the minimum width to height ratio of the channels (225CH) of the one or more airflow straightening structures (225) ranges from 1:1.6 to 1:5.
Embodiment 142. The device or disposable cartridge (200) of the preceding Embodiment, where the minimum width to height ratio of the channels (225CH) of the one or more airflow straightening structures (225) ranges from 1:1.8 to 1:4.
Embodiment 143. The device or disposable cartridge (200) of the preceding Embodiment, where the minimum width to height ratio of the channels (225CH) of the one or more airflow straightening structures (225) ranges from 1:1.9 to 1:3.
Embodiment 144. The device or disposable cartridge (200) of the Embodiments 1 to Embodiment 13, Embodiment 15, Embodiment 18, Embodiment 21, Embodiment 22, Embodiment 24 to Embodiment 25 or Embodiment 27 to Embodiment 142 where the centerlines of any two closer channels (225CH) of the one or more airflow straightening structures (225) are from 1 to 5 mm apart from each other measured from centerline to centerline at the downstream mouth of the channels (225CH).
Embodiment 145. The device or disposable cartridge (200) of the preceding Embodiment, where the centerlines of any two closer channels (225CH) of the one or more airflow straightening structures (225) are from 1.1 to 4.5 mm apart.
Embodiment 146. The device or disposable cartridge (200) of the preceding Embodiment, where the centerlines of any two closer channels (225CH) of the one or more airflow straightening structures (225) are from 1.2 to 4 mm apart.
Embodiment 147. The device or disposable cartridge (200) of the preceding Embodiment, where the centerlines of any two closer channels (225CH) of the one or more airflow straightening structures (225) are from 1.3 to 3.75 mm apart.
Embodiment 148. The device or disposable cartridge (200) of the preceding Embodiment, where the centerlines of any two closer channels (225CH) of the one or more airflow straightening structures (225) are from 1.4 to 3.5 mm apart.
Embodiment 149. The device or disposable cartridge (200) of the preceding Embodiment, where the centerlines of any two closer channels (225CH) of the one or more airflow straightening structures (225) are from 1.5 to 3.25 mm apart.
Embodiment 150. The device or disposable cartridge (200) of the preceding Embodiment, where the centerlines of any two closer channels (225CH) of the one or more airflow straightening structures (225) are from 1.6 to 3 mm apart.
Embodiment 151. The device or disposable cartridge (200) of the preceding Embodiment, where the centerlines of any two closer channels (225CH) of the one or more airflow straightening structures (225) are from 1.7 to 2.9 mm apart.
Embodiment 152. The device or disposable cartridge (200) of the preceding Embodiment, where the centerlines of any two closer channels (225CH) of the one or more airflow straightening structures (225) are from 1.8 to 2.8 mm apart.
Embodiment 153. The device or disposable cartridge (200) of the preceding Embodiment, where the centerlines of any two closer channels (225CH) of the one or more airflow straightening structures (225) are from 1.9 to 2.7 mm apart.
Embodiment 154. The device or disposable cartridge (200) of the preceding Embodiment, where the centerlines of any two closer channels (225CH) of the one or more airflow straightening structures (225) are from 2.0 to 2.6 mm apart.
Embodiment 155. The device or disposable cartridge (200) of any of the Embodiments 1 to Embodiment 13, Embodiment 15, Embodiment 18, Embodiment 21, Embodiment 22, Embodiment 24, Embodiment 25 or Embodiment 27 to Embodiment 153, where the one or more airflow straightening structures (225) include between 5 and 20 channels (225CH).
Embodiment 156. The device or disposable cartridge (200) of the preceding Embodiment, where the one or more airflow straightening structures (225) include between 6 and 17 channels (225CH).
Embodiment 157. The device or disposable cartridge (200) of the preceding Embodiment, where the one or more airflow straightening structures (225) include between 7 and 16 channels (225CH).
Embodiment 158. The device or disposable cartridge (200) of the preceding Embodiment, where the one or more airflow straightening structures (225) include between 8 and 15 channels (225CH).
Embodiment 159. The device or disposable cartridge (200) of the preceding Embodiment, where the one or more airflow straightening structures (225) include between 9 and 14 channels (225CH).
Embodiment 160. The device or disposable cartridge (200) of any of the Embodiments 1 to Embodiment 13, Embodiment 15, Embodiment 18, Embodiment 21, Embodiment 22, Embodiment 24, Embodiment 25 or Embodiment 27 to Embodiment 158, where the centerlines of the channels (225CH) of the one or more airflow straightening structures (225) are within 25% to 75% of the internal height of the handheld medical device immediately downstream of the one or more airflow straightening structures (225).
Embodiment 161. The device or disposable cartridge (200) of the preceding Embodiment, where the centerlines of the channels (225CH) of the one or more airflow straightening structures (225) are within 33% to 66% of the internal height of the handheld medical device immediately downstream of the one or more airflow straightening structures (225).
Embodiment 162. The device or disposable cartridge (200) of the preceding Embodiment, where the centerlines of the channels (225CH) of the one or more airflow straightening structures (225) are within 37% to 63% of the internal height of the handheld medical device immediately downstream of the one or more airflow straightening structures (225).
Embodiment 163. The device or disposable cartridge (200) of any of the Embodiments 1 to Embodiment 13, Embodiment 15, Embodiment 18, Embodiment 21, Embodiment 22, Embodiment 24, Embodiment 25 or Embodiment 27 to Embodiment 161, where the centerlines of the channels (225CH) of the one or more airflow straightening structures (225) are within 5% to 33% and/or 95% to 66% of the internal height of the handheld medical device measured immediately downstream of the one or more airflow straightening structures (225).
Embodiment 164. The device or disposable cartridge (200) of the preceding Embodiment, where the centerlines of the channels (225CH) of the one or more airflow straightening structures (225) are within 10% to 25% and/or 90 to 75% of the internal height of the handheld medical device measured immediately downstream of the one or more airflow straightening structures (225).
Embodiment 165. The device or disposable cartridge (200) of the preceding Embodiment, where the centerlines of the channels (225CH) of the one or more airflow straightening structures (225) are within 10% to 20% and/or 90% to 80% of the internal height of the handheld medical device measured immediately downstream of the one or more airflow straightening structures (225).
Embodiment 166. The device or disposable cartridge (200) of the preceding Embodiment, where the centerlines of the channels (225CH) of the one or more airflow straightening structures (225) are within 10% to 15% and/or 90% to 85% of the internal height of the handheld medical device measured immediately downstream of the one or more airflow straightening structures (225).
Embodiment 167. The device or disposable cartridge (200) of any of the Embodiments 1 to Embodiment 13, Embodiment 15, Embodiment 18, Embodiment 21, Embodiment 22, Embodiment 24, Embodiment 25 or Embodiment 27 to Embodiment 166, where at least part of the channels (225CH) of the one or more airflow straightening structures (225) are connected by a slit (225SL).
Embodiment 168. The device or disposable cartridge (200) of the preceding Embodiment, where the slit (225SL) forms a −10° to 10° angle with the aerosol axis-air inlet plane, which is the plane defined by the aerosol axis (223) and the center of the air inlet (220).
Embodiment 169. The device or disposable cartridge (200) of the preceding Embodiment, where the slit (225SL) forms a −5° to 5° angle with the aerosol axis-air inlet plane.
Embodiment 170. The device or disposable cartridge (200) of the preceding Embodiment, where the slit (225SL) forms a −2° to 2° angle with the aerosol axis-air inlet plane.
Embodiment 171. The device or disposable cartridge (200) of the Embodiment 166 to Embodiment 169, where the slit (225SL) ranges from 0.1 to 1.5 mm high.
Embodiment 172. The device or disposable cartridge (200) of the preceding Embodiment, where the slit (225SL) ranges from 0.12 to 1.25 mm high.
Embodiment 173. The device or disposable cartridge (200) of the preceding Embodiment, where the slit (225SL) ranges from 0.15 to 1 mm high.
Embodiment 174. The device or disposable cartridge (200) of the preceding Embodiment, where the slit (225SL) ranges from 0.17 to 0.75 mm high.
Embodiment 175. The device or disposable cartridge (200) of the preceding Embodiment, where the slit (225SL) ranges from 0.2 to 0.5 mm high.
Embodiment 176. The device or disposable cartridge (200) of the preceding Embodiment, where the slit (225SL) ranges from 0.2 to 0.4 mm high.
Embodiment 177. The device or disposable cartridge (200) of any of the Embodiments 1 to Embodiment 13, Embodiment 15, Embodiment 18, Embodiment 21, Embodiment 22, Embodiment 24, Embodiment 25 or Embodiment 27 to Embodiment 175, where both most lateral channels (225CH) of the one or more airflow straightening structures (225) are up to four times wider than the channels (225CH) of the one or more airflow straightening structures (225).
Embodiment 178. The device or disposable cartridge (200) of any of the Embodiments 1 to Embodiment 13, Embodiment 15, Embodiment 18, Embodiment 21, Embodiment 22, Embodiment 24, Embodiment 25 or Embodiment 27 to Embodiment 176, where the one or more airflow straightening structures (225) include cavities (225C) upstream and/or downstream the channels (225CH).
Embodiment 179. The device or disposable cartridge (200) of the preceding Embodiment, where the channels (225CH) and the cavities (225C) are
   a. H shaped,
   b. right tack shaped (├), or
   c. left tack shaped (┤); and
      the channels (225CH) being the crossbar (C) of the H; ├ or ┤, and the cavities (225C) being the stems(S) of the H, ├ or ┤; and the cavities (225C) extending up to 100% high of the internal height of the device the closest of just downstream or upstream of the one or more airflow straightening structures (225).
Embodiment 180. The device or disposable cartridge (200) of the preceding Embodiment, where the crossbar-stems plane forms an angle from 80° to 100° with the aerosol axis-air inlet plane, which is the plane defined by the aerosol axis (223) and the center of the air inlet (220).
Embodiment 181. The device or disposable cartridge (200) of the preceding Embodiment, where the crossbar-stems plane forms an angle from 85° to 95° with the aerosol axis-air inlet plane.
Embodiment 182. The device or disposable cartridge (200) of any of the Embodiment 177 to Embodiment 180, where the cavities (225C) extend 0.5 to 4 mm upstream (U_{A}) and/or downstream (D_{A}) the channels (225CH) of the one or more airflow straightening structures (225).
Embodiment 183. The device or disposable cartridge (200) of any of the preceding Embodiment, where the cavities (225C) extend 0.75 to 3.5 mm upstream (U_{A}) and/or downstream (D_{A}) the channels (225CH) of the one or more airflow straightening structures (225).
Embodiment 184. The device or disposable cartridge (200) of any of the preceding Embodiment, where the cavities (225C) extend 1 to 3.0 mm upstream (U_{A}) and/or downstream (D_{A}) the channels (225CH) of the one or more airflow straightening structures (225).
Embodiment 185. The device or disposable cartridge (200) of any of the preceding Embodiment, where the cavities (225C) extend 1.25 to 2.5 mm upstream (U_{A}) and/or downstream (D_{A}) the channels (225CH) of the one or more airflow straightening structures (225).
Embodiment 186. The device or disposable cartridge (200) of any of the preceding Embodiment, where the cavities (225C) extend 1.5 to 2.5 mm upstream (U_{A}) and/or downstream (D_{A}) the channels (225CH) of the one or more airflow straightening structures (225).
Embodiment 187. The device or disposable cartridge (200) of any of the Embodiment 177 to Embodiment 185, where the cavities (225C), in its narrowest section, are 100 to 125% as wide as the channels (225CH) of the one or more airflow straightening structures (225).
Embodiment 188. The device or disposable cartridge (200) of any of the Embodiments 1 to Embodiment 13, Embodiment 18, Embodiment 24, Embodiment 25 or Embodiment 27 to Embodiment 186, where the channels (224CH) of the one or more airflow diverting structures (224) and the channels (225CH) of the one or more airflow straightening structures (225) define a continuous channel.
Embodiment 189. The device or disposable cartridge (200) of any of the Embodiments 1 to Embodiment 13, Embodiment 16, Embodiment 19, Embodiment 21,
Embodiment 23, Embodiment 24 or Embodiment 26 to Embodiment 187, where the angle δ is between 7.5 and 15°.
Embodiment 190. The device or disposable cartridge (200) of the preceding Embodiment, where the angle δ is between 8 and 12°.
Embodiment 191. The device or disposable cartridge (200) of any of the Embodiments 1 to Embodiment 13, Embodiment 16, Embodiment 19, Embodiment 21, Embodiment 23, Embodiment 24, Embodiment 26 to Embodiment 189, where the internal opposite walls (222, 222a, 222b) are straight, convex or concave.
Embodiment 192. The device or disposable cartridge (200) of the preceding Embodiment, where the internal opposite walls (222) are straight.
Embodiment 193. The device or disposable cartridge (200) of Embodiment 14, where the internal opposite (222a) walls are convex.
Embodiment 194. The device or disposable cartridge (200) of Embodiment 14, where the internal opposite walls (222b) are concave.
Embodiment 195. The device or disposable cartridge (200) of any of the Embodiments 1 to Embodiment 13, Embodiment 16, Embodiment 19, Embodiment 21, Embodiment 23, Embodiment 24 or Embodiment 26 to Embodiment 193, where the walls define an angle δ in at least 90% of its portion included within the projection (Pf) of the drug foil substrate (205) on the aerosol axis (223).
Embodiment 196. The device or disposable cartridge (200) of the preceding Embodiment, where the walls define an angle δ in at least 100% of its portion included within the projection (Pf) of the drug foil substrate (205) on the aerosol axis (223).
Embodiment 197. The device or disposable cartridge (200) of the preceding Embodiment, where the walls define an angle δ in at least 110% of its portion included within the projection (Pf) of the drug foil substrate (205) on the aerosol axis (223).
Embodiment 198. The device or disposable cartridge (200) of the preceding Embodiment, where the walls define an angle δ in at least 125% of its portion included within the projection (Pf) of the drug foil substrate (205) on the aerosol axis (223).
Embodiment 199. The device or disposable cartridge (200) of any of the Embodiments 1 to Embodiment 13, Embodiment 17, Embodiment 20, Embodiment 22, Embodiment 23, or Embodiment 25 to Embodiment 197, where the angle γ ranges from 10° to 20°.
Embodiment 200. The device or disposable cartridge (200) of the preceding Embodiment, where the angle γ ranges from 12° to 18°.
Embodiment 201. The device or disposable cartridge (200) of the preceding Embodiment, where the angle γ ranges from 13° to 17°.
Embodiment 202. The device or disposable cartridge (200) of any of the Embodiments 1 to Embodiment 13, Embodiment 17, Embodiment 20, Embodiment 22, Embodiment 23, Embodiment 25 to Embodiment 200, where the channels (226CHL and 226CHR) of the one or more airflow directing structures (226) are curved and the angle γ is measured using the axis defined by the center of the upstream mouth of the channel (226CHL or 226CHR) and the center of the downstream mouth of the channel (226CHL or 226CHR) instead of the centerline.
Embodiment 203. The device or disposable cartridge (200) of any of the Embodiments 1 to Embodiment 13, Embodiment 17, Embodiment 20, Embodiment 22, Embodiment 23, Embodiment 25 to Embodiment 201, where the channels (226CHL and 226CHR) of the one or more airflow directing structures (226) extend from 0.7 to 5 mm measured in the centerline direction.
Embodiment 204. The device or disposable cartridge (200) of the preceding Embodiment, where the channels (226CHL and 226CHR) of the one or more airflow directing structures (226) extend from 1 to 4.5 mm.
Embodiment 205. The device or disposable cartridge (200) of the preceding Embodiment, where the channels (226CHL and 226CHR) of the one or more airflow directing structures (226)) extend from 1.2 to 4 mm.
Embodiment 206. The device or disposable cartridge (200) of the preceding Embodiment, where the channels (226CHL and 226CHR) of the one or more airflow directing structures (226) extend from 1.3 to 3.5 mm.
Embodiment 207. The device or disposable cartridge (200) of the preceding Embodiment, where the channels (226CHL and 226CHR) of the one or more airflow directing structures (226) extend from 1.4 to 3.5 mm.
Embodiment 208. The device or disposable cartridge (200) of the preceding Embodiment, where the channels (226CHL and 226CHR) of the one or more airflow directing structures (226) extend from 1.5 to 3.5 mm.
Embodiment 209. The device or disposable cartridge (200) of the preceding Embodiment, where the channels (226CHL and 226CHR) of the one or more airflow directing structures (226) extend from 1.6 to 3.5 mm.
Embodiment 210. The device or disposable cartridge (200) of any of the Embodiments 1 to Embodiment 13, Embodiment 17, Embodiment 20, Embodiment 22, Embodiment 23, Embodiment 25 to Embodiment 208, where minimum width of the channels (226CHL and 226CHR) of the one or more airflow directing structures (226) ranges from 0.3 to 5 mm.
Embodiment 211. The device or disposable cartridge (200) of the preceding Embodiment, where the minimum width of the channels (226CHL and 226CHR) of the one or more airflow directing structures (226) ranges from 0.35 to 4 mm.
Embodiment 212. The device or disposable cartridge (200) of the preceding Embodiment, where the minimum width of the channels (226CHL and 226CHR) of the one or more airflow directing structures (226) ranges from 0.4 to 3 mm.
Embodiment 213. The device or disposable cartridge (200) of the preceding Embodiment, where the minimum width of the channels (226CHL and 226CHR) of the one or more airflow directing structures (226) ranges from 0.4 to 2 mm.
Embodiment 214. The device or disposable cartridge (200) of the preceding Embodiment, where the minimum width of the channels (226CHL and 226CHR) of the one or more airflow directing structures (226) ranges from 0.4 to 1.5 mm.
Embodiment 215. The device or disposable cartridge (200) of the preceding Embodiment, where the minimum width of the channels (226CHL and 226CHR) of the one or more airflow directing structures (226) ranges from 0.5 to 1.5 mm.
Embodiment 216. The device or disposable cartridge (200) of the preceding Embodiment, where the minimum width of the channels (226CHL and 226CHR) of the one or more airflow directing structures (226) ranges from 0.7 to 1.25 mm.
Embodiment 217. The device or disposable cartridge (200) of the Embodiments 1 to Embodiment 13, Embodiment 17, Embodiment 20, Embodiment 22, Embodiment 23, Embodiment 25 to Embodiment 215, where the minimum height of the channels (226CHL and 226CHR) of the one or more airflow directing structures (226) ranges from 3 to 15 mm.
Embodiment 218. The device or disposable cartridge (200) of the preceding Embodiment, where the minimum height of the channels (226CHL and 226CHR) of the one or more airflow directing structures (226) ranges from 10 to 15 mm.
Embodiment 219. The device or disposable cartridge (200) of the preceding Embodiment, where the minimum height of the channels (226CHL and 226CHR) of the one or more airflow directing structures (226) ranges from 11 to 14 mm.
Embodiment 220. The device or disposable cartridge (200) of the Embodiments 1 to Embodiment 13, Embodiment 17, Embodiment 20, Embodiment 22, Embodiment 23, Embodiment 25 to Embodiment 218, where the centerlines of any two closer channels (226CHL or 226CHR) of the one or more airflow directing structures (226) are from 0.5 to 3 mm apart from each other measured from centerline to centerline at the downstream mouth of the channels (226CHL or 226CHR).
Embodiment 221. The device or disposable cartridge (200) of the preceding Embodiment, where the centerlines of any two closer channels (226CHL or 226CHR) of the one or more airflow directing structures (226) are from 0.75 to 2.5 mm apart.
Embodiment 222. The device or disposable cartridge (200) of the preceding Embodiment, where the centerlines of any two closer channels (226CHL or 226CHR) of the one or more airflow directing structures (226) are from 1 to 2 mm apart.
Embodiment 223. The device or disposable cartridge (200) of the preceding Embodiment, where the centerlines of any two closer channels (226CHL or 226CHR) of the one or more airflow directing structures (226) are from 1.25 to 1.75 mm apart.
Embodiment 224. The device or disposable cartridge (200) of any of the Embodiments 1 to Embodiment 13, Embodiment 17, Embodiment 20, Embodiment 22, Embodiment 23, Embodiment 25 to Embodiment 222, where the one or more airflow directing structures (226) include between 5 and 20 channels (226CHL and 226CHR).
Embodiment 225. The device or disposable cartridge (200) of the preceding Embodiment, where the one or more airflow directing structures (226) include between 5 and 15 channels (226CHL and 226CHR).
Embodiment 226. The device or disposable cartridge (200) of the preceding Embodiment, where the one or more airflow directing structures (226) include between 6 and 12 channels (226CHL and 226CHR).
Embodiment 227. The device or disposable cartridge (200) of any of the Embodiments 1 to Embodiment 13, Embodiment 17, Embodiment 20, Embodiment 22, Embodiment 23, Embodiment 25 to Embodiment 225, where the centerlines of the channels (226CHL and 226CHR) of the one or more airflow directing structures (226) are within 25% to 75% of the internal height of the handheld medical device measured immediately downstream of the one or more airflow directing structures (226).
Embodiment 228. The device or disposable cartridge (200) of the preceding Embodiment, where the centerlines of the channels (226CHL and 226CHR) of the one or more airflow directing structures (226) are within 33% to 66% of the internal height of the handheld medical device measured immediately downstream of the one or more airflow directing structures (226).
Embodiment 229. The device or disposable cartridge (200) of the preceding Embodiment, where the centerlines of the channels (226CHL and 226CHR) of the one or more airflow directing structures (226) are within 37% to 63% of the internal height of the handheld medical device measured immediately downstream of the one or more airflow directing structures (226).
Embodiment 230. The device or disposable cartridge (200) of any of the Embodiments 1 to Embodiment 13, Embodiment 17, Embodiment 20, Embodiment 22, Embodiment 23, Embodiment 25 to Embodiment 225, where the centerlines of the channels (226CHL and 226CHR) of the one or more airflow directing structures (226) are within 5% to 33% and/or 95% to 66% of the internal height of the handheld medical device measured immediately downstream of the one or more airflow directing structures (226).
Embodiment 231. The device or disposable cartridge (200) of the preceding Embodiment, where the centerlines of the channels (226CHL and 226CHR) of the one or more airflow directing structures (226). are within 10% to 25% and/or 90 to 75% of the internal height of the handheld medical device measured immediately downstream of the one or more airflow directing structures (226).
Embodiment 232. The device or disposable cartridge (200) of the preceding Embodiment, where the centerlines of the channels (226CHL and 226CHR) of the one or more airflow directing structures (226). are within 10% to 20% and/or 90% to 80% of the internal height of the handheld medical device measured immediately downstream of the one or more airflow directing structures (226).
Embodiment 233. The device or disposable cartridge (200) of the preceding Embodiment, where the centerlines of the channels (226CHL and 226CHR) of the one or more airflow directing structures (226). are within 10% to 15% and/or 90% to 85% of the internal height of the handheld medical device measured immediately downstream of the one or more airflow directing structures (226).
Embodiment 234. The device or disposable cartridge (200) of any of the Embodiments 1 to Embodiment 13, Embodiment 17, Embodiment 20, Embodiment 22, Embodiment 23, Embodiment 25 to Embodiment 232, where at least part of the channels (226CHL and 226CHR) of the one or more airflow directing structures (226) are connected by a slit.
Embodiment 235. The device or disposable cartridge (200) of the preceding Embodiment, where the central plane of the slit forms a −10° to 10° angle with the aerosol axis-air inlet plane, which is the plane defined by the aerosol axis (223) and the center of the air inlet (220).
Embodiment 236. The device or disposable cartridge (200) of the preceding Embodiment, where the central plane of the slit forms a −5° to 5° angle with the aerosol axis-air inlet plane.
Embodiment 237. The device or disposable cartridge (200) of the preceding Embodiment, where the central plane of the slit forms a −2° to 2° angle with the aerosol axis-air inlet plane.
Embodiment 238. The device or disposable cartridge (200) of the Embodiment 233 to Embodiment 237, where the slit ranges from 0.1 to 3 mm high.
Embodiment 239. The device or disposable cartridge (200) of the preceding Embodiment, where the slit ranges from 0.25 to 2.5 mm high.
Embodiment 240. The device or disposable cartridge (200) of the preceding Embodiment, where the slit ranges from 0.5 to 2.25 mm high.
Embodiment 241. The device or disposable cartridge (200) of the preceding Embodiment, where the slit ranges from 0.75 to 2 mm high.
Embodiment 242. The device or disposable cartridge (200) of the preceding Embodiment, where the slit ranges from 1 to 2 mm high.
Embodiment 243. The device or disposable cartridge (200) of the preceding Embodiment, where the slit ranges from 1.25 to 1.75 mm high.
Embodiment 244. The device or disposable cartridge (200) of any of the Embodiments 1 to Embodiment 13, Embodiment 17, Embodiment 20, Embodiment 22, Embodiment 23, Embodiment 25 to Embodiment 242, where most lateral channels of the one or more airflow directing structures (226) are up to four times wider than the rest of the channels (226CHL and 226CHR) of the one or more airflow directing structures (226).
Embodiment 245. The device or disposable cartridge (200) of any of the Embodiments 1 to Embodiment 13, Embodiment 17, Embodiment 20, Embodiment 22, Embodiment 23, Embodiment 25 to Embodiment 243, where the one or more airflow directing structures (226) include cavities upstream and/or downstream the channels (226CHL and 226CHR).
Embodiment 246. The device or disposable cartridge (200) of the preceding Embodiment, where the channels (226CHL and 226CHR) and the cavities are
   a. H shaped,
   b. right tack shaped (├), or
   c. left tack shaped (┤); and
      the channels (226CHL and 226CHR) being the crossbar (C) of the H, ├ or ┤; the cavities being the stems(S) of the H, ├ or ┤; and the cavities extending up to 100% of the internal height of the device the closest of just downstream or upstream of the one or more airflow directing structures (226).
Embodiment 247. The device or disposable cartridge (200) of the preceding Embodiment, where the crossbar-stems plane forms an angle from 80° to 100° with the aerosol axis-air inlet plane, which is the plane defined by the aerosol axis (223) and the center of the air inlet (220).
Embodiment 248. The device or disposable cartridge (200) of the preceding Embodiment, where the crossbar-stems plane forms an angle from 85° to 95° with the aerosol axis-air inlet plane.
Embodiment 249. The device or disposable cartridge (200) of any of the Embodiment 244 to Embodiment 247, where the cavities extend from 0.1 to 2.5 mm upstream (U_{A}) and/or downstream (D_{A}) the channels (226CHL and 226CHR) of the one or more airflow directing structures (226) measured in the centerline direction.
Embodiment 250. The device or disposable cartridge (200) of the preceding Embodiment, where the cavities extend from 0.15 to 2 mm upstream (U_{A}) and/or downstream (D_{A}) the channels (226CHL and 226CHR) of the one or more airflow directing structures (226) measured in the centerline direction
Embodiment 251. The device or disposable cartridge (200) of the preceding Embodiment, where the cavities extend from 0.2 to 1.5 mm upstream (U_{A}) and/or downstream (D_{A}) the channels (226CHL and 226CHR) of the one or more airflow directing structures (226) measured in the centerline direction
Embodiment 252. The device or disposable cartridge (200) of the preceding Embodiment, where the cavities extend from 0.25 to 1 mm upstream (U_{A}) and/or downstream (D_{A}) the channels (226CHL and 226CHR) of the one or more airflow directing structures (226) measured in the centerline direction
Embodiment 253. The device or disposable cartridge (200) of the preceding Embodiment, where the cavities extend from 0.3 to 0.75 mm upstream (U_{A}) and/or downstream (D_{A}) the channels (226CHL and 226CHR) of the one or more airflow directing structures (226) measured in the centerline direction.
Embodiment 254. The device or disposable cartridge (200) of any of the Embodiment 244 to Embodiment 252, where the width of the cavities, in its narrowest section, are 100 to 125% as width as the channels (226CHL and 226CHR) of the one or more airflow directing structures (226).
Embodiment 255. The device or disposable cartridge (200) of any of the preceding Embodiments, where the solid drug film (207) coated on the drug foil substrate (205) has an area density from 0.001 to 5 mg/cm².
Embodiment 256. The device or disposable cartridge (200) of the preceding Embodiment, where the solid drug film (207) coated on the drug foil substrate (205) has an area density from 0.005 to 4 mg/cm².
Embodiment 257. The device or disposable cartridge (200) of the preceding Embodiment, where the solid drug film (207) coated on the drug foil substrate (205) has an area density from 0.01 to 3 mg/cm².
Embodiment 258. The device or disposable cartridge (200) of the preceding Embodiment, where the solid drug film (207) coated on the drug foil substrate (205) has an area density from 0.05 to 2.5 mg/cm².
Embodiment 259. The device or disposable cartridge (200) of the preceding Embodiment, where the solid drug film (207) coated on the drug foil substrate (205) has an area density from 0.1 to 2 mg/cm².
Embodiment 260. The device or disposable cartridge (200) of any of the preceding Embodiments, where the solid drug film is coated on a portion of the drug foil substrate (205) in a trapezoidal shape, a crescent shape, a circular shape, a rectangular shape or a square shape.
Embodiment 261. The device or disposable cartridge (200) of any of the preceding Embodiments, where the longitudinal axis of the drug foil substrate defines an angle of −10° to 10° with the aerosol axis (223).
Embodiment 262. The device or disposable cartridge (200) of the preceding Embodiment, where the longitudinal axis of the drug foil substrate defines an angle of −5° to 5° with the aerosol axis (223).
Embodiment 263. The device or disposable cartridge (200) of the preceding Embodiment, where the longitudinal axis of the drug foil substrate defines an angle of −2° to 2° with the aerosol axis (223).
Embodiment 264. The device or disposable cartridge (200) of any of the preceding Embodiments, where the lateral axis of the drug foil substrate defines an angle of −10° to 10° with the aerosol axis-air inlet plane.
Embodiment 265. The device or disposable cartridge (200) of the preceding Embodiment, where the lateral axis of the drug foil substrate defines an angle of −5° to 5° with the aerosol axis-air inlet plane.
Embodiment 266. The device or disposable cartridge (200) of the preceding Embodiment, where the lateral axis of the drug foil substrate defines an angle of −2° to 2° with the aerosol axis-air inlet plane.
Embodiment 267. The device or disposable cartridge (200) of any of the preceding Embodiments, where the drug is selected from loxapine, alprazolam, estazolam, fentanyl, zaleplon, almorexant, apomorphine, pergolide, ropinirole, pramipexole, granisetron, ondansetron, palonosetron, nicotine, rotigotine, or its pharmaceutically acceptable salts.
Embodiment 268. The device or disposable cartridge (200) of any of the preceding Embodiments, where the condensation aerosol particles have a MMAD (Median Mass Aerodynamic Diameter) from 0.2 to 5 μm measured using a next generation impactor technique.
Embodiment 269. The device or disposable cartridge (200) of the preceding Embodiment, where the condensation aerosol particles have a MMAD (Median Mass Aerodynamic Diameter) from 0.3 to 4 μm.
Embodiment 270. The device or disposable cartridge (200) of the preceding Embodiment, where the condensation aerosol particles have a MMAD (Median Mass Aerodynamic Diameter) from 0.4 to 3 μm.
Embodiment 271. The device or disposable cartridge (200) of the preceding Embodiment, where the condensation aerosol particles have a MMAD (Median Mass Aerodynamic Diameter) from 0.5 to 2 μm.
Embodiment 272. The device or disposable cartridge (200) of the preceding Embodiment, where the condensation aerosol particles have a MMAD (Median Mass Aerodynamic Diameter) from 0.6 to 1.5 μm.
Embodiment 273. The device or disposable cartridge (200) of any of the preceding Embodiments, where the drug foil substrate (205) is metallic.
Embodiment 274. The device or disposable cartridge (200) of any of the preceding Embodiments, where the drug foil substrate (205) is made of stainless-steel.
Embodiment 275. The device or disposable cartridge (200) of any of the preceding Embodiments, suitable to deliver a single dose.
Embodiment 276. The device or disposable cartridge (200) of any of the Embodiments 1 to Embodiment 273 which is a multi-dose cartridge.
Embodiment 277. The device or disposable cartridge (200) of any of the preceding Embodiments, which externally is 60-230 mm×40-90 mm×9-50 mm, or in another embodiment is 60-140 mm×40-75 mm×9-25 mm or in another embodiment is 85-125 mm×50-65 mm×9-20 mm.
Embodiment 278. The device or disposable cartridge (200) of any of the Embodiments 1 or Embodiment 7 to Embodiment 276, where the drug foil substrate heating circuit includes a chemical heat pack.
Embodiment 279. The device or disposable cartridge (200) of the preceding Embodiment, where the chemical heat pack is based on an exothermic chemical reaction, such as exothermic solvation, hydration of pyrophoric materials and oxidation of combustible materials.
Embodiment 280. The device or disposable cartridge (200) of any of the preceding Embodiments, which does not include any cooling element.
Embodiment 281. The device or disposable cartridge (200) of any of the preceding Embodiments, where the only heat source is the drug foil substrate heating circuit to vaporize the solid drug film coated on at least a portion of the drug foil substrate (205).
Embodiment 282. The device or disposable cartridge (200) of any of the preceding Embodiments, where at least one internal wall of the airway (203) includes antistatic material.
Embodiment 283. The device or disposable cartridge (200) of any of the preceding Embodiments, where the drug foil substrate support (204) is totally or partially placed within the airway (203).
Embodiment 284. The device or disposable cartridge (200) of any of the preceding Embodiments, where the drug foil substrate support (204) is totally or partially placed outside the airway (203).
Embodiment 285. The device or disposable cartridge (200) of any of the preceding Embodiments, where the drug foil substrate support (204) includes one or more of the walls defining the airway (203).
Embodiment 286. The device or disposable cartridge (200) of any of the preceding Embodiments, where any of the diverting channels (224CH), straightening channels (225CH) and/or airflow directing channels (226CHL and 226CHR) have a straight shape.
Embodiment 287. The device or disposable cartridge (200) of any of the Embodiment 1 to Embodiment 284, where any of the diverting channels (224CH), straightening channels (225CH) and/or airflow directing channels (226CHL and 226CHR) have an elliptical shape.
Embodiment 288. The device or disposable cartridge (200) of any of the preceding Embodiments, where the airflow detector triggers the heating of the drug foil substrate at an airflow higher than 7 L/min.
Embodiment 289. The device or disposable cartridge (200) of the preceding Embodiment, where the airflow detector triggers the heating of the drug foil substrate at an airflow higher than 14 L/min.
Embodiment 290. The device or disposable cartridge (200) of the preceding Embodiment, where the airflow detector triggers the heating of the drug foil substrate at an airflow higher than 20 L/min.
Embodiment 291. The device or disposable cartridge (200) of the preceding Embodiment, where the airflow detector triggers the heating of the drug foil substrate at an airflow higher than 30 L/min.
Embodiment 292. The device or disposable cartridge (200) of any of the preceding Embodiments, where the air inlet (220) is in fluid communication with the air flow detector.
Embodiment 293. The handheld medical device or disposable cartridge (200) of any of the preceding Embodiments for use in therapy.
Embodiment 294. The device or disposable cartridge for use in therapy according to Embodiment 292, where when the drug in the solid drug film (207) is:
   a. loxapine or its pharmaceutically acceptable salts, the condition or episode is agitation, including:
      I. rapidly control mild to moderate agitation in adults with schizophrenia or bipolar disorder, or
      II. acute agitation associated with schizophrenia or bipolar disorder in adults;
   b. alprazolam, estazolam or its pharmaceutically acceptable salts, the condition or episode is epilepsy, where epilepsy includes seizures;
   c. fentanyl or its pharmaceutically acceptable salts, the condition or episode is breakthrough pain;
   d. zaleplon, almorexant or its pharmaceutically acceptable salts, the condition or episode is a sleep disorder including:
      I. middle of the night awakening, or
      II. middle of the night insomnia;
   e. apomorphine, pergolide, ropinirole, pramipexole, or its pharmaceutically acceptable salts, the condition or episode is Parkinson's disease, off-episodes in Parkinson's disease, and/or idiopathic Parkinson's disease;
   f. granisetron, ondansetron, palonosetron or its pharmaceutically acceptable salts, the condition or episode is:
      I. nausea,
      II. vomiting or
      III. cyclic vomiting syndrome;
   g. nicotine or its pharmaceutically acceptable salts including nicotine meta-salicylate, the condition or episode is nicotine craving and/or effecting cessation of smoking; or
   h. ropinirole, pramipexole, or rotigotine, the condition or episode is restless legs syndrome.
Embodiment 295. A method of treatment for a condition or episode which includes administering a drug included in the solid drug film (207) coated on at least a portion of the drug foil substrate (205) of the handheld medical device or disposable cartridge (200) of any of the Embodiments 1 to Embodiment 291, where when the drug is:
   a. loxapine or its pharmaceutically acceptable salts, the condition or episode is agitation, including:
      I. rapidly control mild to moderate agitation in adults with schizophrenia or bipolar disorder, or
      II. acute agitation associated with schizophrenia or bipolar disorder in adults;
   b. alprazolam, estazolam or its pharmaceutically acceptable salts, the condition or episode is epilepsy, where epilepsy includes seizures;
   c. fentanyl or its pharmaceutically acceptable salts, the condition or episode is breakthrough pain;
   d. zaleplon, almorexant or its pharmaceutically acceptable salts, the condition or episode is a sleep disorder including:
      I. middle of the night awakening, or
      II. middle of the night insomnia;
   e. apomorphine, pergolide, ropinirole, pramipexole, or its pharmaceutically acceptable salts, the condition or episode is Parkinson's disease, off-episodes in Parkinson's disease, and/or idiopathic Parkinson's disease;
   f. granisetron, ondansetron, palonosetron or its pharmaceutically acceptable salts, the condition or episode is:
      I. nausea,
      II. vomiting or
      III. cyclic vomiting syndrome;
   g. nicotine or its pharmaceutically acceptable salts including nicotine meta-salicylate, the condition or episode is nicotine craving and/or effecting cessation of smoking; or
   h. ropinirole, pramipexole, or rotigotine, the condition or episode is restless legs syndrome.
Embodiment 296. The use of a handheld medical device or disposable cartridge (200) according to any of the Embodiments 1 to Embodiment 291 which includes a drug included in the solid drug film coated on at least a portion of the drug foil substrate (205) for the manufacturing of a medicament for the treatment of a condition or episode; where when the drug is:
   a. loxapine or its pharmaceutically acceptable salts, the condition or episode is agitation, including:
      I. rapidly control mild to moderate agitation in adults with schizophrenia or bipolar disorder, or
      II. acute agitation associated with schizophrenia or bipolar disorder in adults;
   b. alprazolam, estazolam or its pharmaceutically acceptable salts, the condition or episode is epilepsy, where epilepsy includes seizures;
   c. fentanyl or its pharmaceutically acceptable salts, the condition or episode is breakthrough pain;
   d. zaleplon, almorexant or its pharmaceutically acceptable salts, the condition or episode is a sleep disorder including:
      I. middle of the night awakening, or
      II. middle of the night insomnia;
   e. apomorphine, pergolide, ropinirole, pramipexole, or its pharmaceutically acceptable salts, the condition or episode is Parkinson's disease, off-episodes in Parkinson's disease, and/or idiopathic Parkinson's disease;
   f. granisetron, ondansetron, palonosetron or its pharmaceutically acceptable salts, the condition or episode is:
      I. nausea,
      II. vomiting or
      III. cyclic vomiting syndrome;
   g. nicotine or its pharmaceutically acceptable salts including nicotine meta-salicylate, the condition or episode is nicotine craving and/or effecting cessation of smoking; or
   h. ropinirole, pramipexole, or rotigotine, the condition or episode is restless legs syndrome.
Embodiment 297. A medicament including a drug selected from: loxapine, alprazolam, estazolam, fentanyl, zaleplon, almorexant, apomorphine, pergolide, ropinirole, pramipexole, granisetron, ondansetron, palonosetron, nicotine, nicotine meta-salicylate, rotigotine, or its pharmaceutically acceptable salts for use in the handheld medical device or disposable cartridge (200) of any of the Embodiments 1 to Embodiment 291.
Embodiment 298. A drug deposited on a drug foil substrate (205) of the handheld medical device or disposable cartridge (200) of any of the Embodiments 1 to Embodiment 291 for use in a condition or episode, where when the drug is
   a. loxapine or its pharmaceutically acceptable salts, the condition or episode is agitation, including:
      I. rapidly control mild to moderate agitation in adults with schizophrenia or bipolar disorder, or
      II. acute agitation associated with schizophrenia or bipolar disorder in adults;
   b. alprazolam, estazolam or its pharmaceutically acceptable salts, the condition or episode is epilepsy, where epilepsy includes seizures;
   c. fentanyl or its pharmaceutically acceptable salts, the condition or episode is breakthrough pain;
   d. zaleplon, almorexant or its pharmaceutically acceptable salts, the condition or episode is a sleep disorder including:
      I. middle of the night awakening, or
      II. middle of the night insomnia;
   e. apomorphine, pergolide, ropinirole, pramipexole, or its pharmaceutically acceptable salts, the condition or episode is Parkinson's disease, off-episodes in Parkinson's disease, and/or idiopathic Parkinson's disease;
   f. granisetron, ondansetron, palonosetron or its pharmaceutically acceptable salts, the condition or episode is:
      I. nausea,
      II. vomiting or
      III. cyclic vomiting syndrome;
   g. nicotine or its pharmaceutically acceptable salts including nicotine meta-salicylate, the condition or episode is nicotine craving and/or effecting cessation of smoking; or
   h. ropinirole, pramipexole, or rotigotine the condition or episode is restless legs syndrome.

### EXAMPLES

### Device Configurations

The table below shows different configurations of the devices' housings:

| Device | A | B | C | D | E | F | G |
|---|---|---|---|---|---|---|---|
| Housing Material | Permastat ®* | Permastat ®* | PA-12** | PA-12** | PA-12** | PA-12** | PA-12** |
| Antistatic | Yes | Yes | No | No | No | No | |
| Heating | Electric | Electric | Electric | Electric | Electric | Electric | Electric |
| Foil substrate | 32 × 32 | 40 × 32 | 40 × 32 | 40 × 32 | 40 × 32 | 40 × 32 | 40 × 32 |
| size(mm) | | | | | | | |
| External housing | 100 | 89.4 | 89.4 | 89.4 | 89.4 | 89.4 | 89.4 |
| length *** (mm) | | | | | | | |
| Bulkhead | Yes | No | No | Yes | No | No | Yes |
| I. airflow diverting | No | Yes | Yes | No | Yes | No | No |
| structure | | | | | | | |
| II. airflow | No | Yes | Yes | No | No | Yes | No |
| straightening | | | | | | | |
| structure | | | | | | | |
| III. internal walls | No | Yes | Yes | No | No | No | Yes |
| forming an angle | | | | | | | |
| Internal walls angle | 0° | 10° | 10° | 0° | 0° | 0° | 10° |
| Such as that in FIG. | 1 | 2A or 18A | 2A, 18A, | 18B or | 18C or | 18D or | 18E or |
| | | | or 21A | 21B | 21C | 21D | 21E |

*polycarbonate;
**nylon;
***excluding air inlet connector

### Method 1: Apomorphine Analysis

Solutions were analysed by UPLC with water (ammonium formate/formic acid) and acetonitrile as mobile phase using UV detection at 275 nm as acquisition wavelength.

### Method 2: Emitted Dose (ED) Testing

This method is based on the US Pharmacopeia “Sampling the Delivered Dose from Metered-Dose Inhalers” of the “Metered-Dose Inhalers and Dry Powder Inhalers” section.

The mouthpiece of the device to be tested was tightly attached to a mouthpiece adapter connected to an end of a 12 cm sample connection tube which at the other end has attached a filter support cap having a ø 47 mm, 2.2 μm pore size, quartz filter (QM-A, Whatman) which was connected to a pump to create an airflow which triggers the flow sensor and the foil substrate is heated.

If the heating is electric, the device was connected, as well, to a controller including batteries and the required electronics to heat the drug foil.

The material in the filter and the sample connection tube was extracted with methanol and analysed as in Method 1.

### Method 3: Particle Size (MMAD) Distribution Using a Next Generation Impactor Technique

Aerosol particles were collected and separated based on their aerodynamic diameter on a NGI Model 170 (NGI, MSP Corporation,) using a methodology based on “Next Generation Pharmaceutical Impactor (NGI)” per US Pharmacopeia <601> and “EP 2.9.18 Preparations for Inhalation: Aerodynamic Assessment of Fine Particles” of the European Pharmacopoeia, 10^{th} edition. The aerosol particles were drawn through successively narrower jets within the impactor and directed against the impaction stages at increasing velocities. Particles with sufficient inertia leave the bent flow streamlines and impact on the collection cups, whereas small particles flow to the next stage.

Drug was recovered from the collection cups by manual extraction. The amount of drug deposited at each stage and component was collected with methanol and was quantified using Method 1

To determine the MMAD, the mass distribution over the particle size effective cut off diameters in the cascade impactor was analysed, and then the median diameter in the distribution at which 50% of the particles by mass are larger and 50% are smaller is the result for the Mass Median Aerodynamic Diameter (MMAD).

### Comparative Example 1: Deposition on Prior Art Devices Actuated by a User

2 mg (for achieving a target Emitted Dose (ED) of 1 mg) or 4 mg (for achieving a target ED of 2 mg) of apomorphine HCl was coated on the foil substrate of Devices A. The heating of the devices was triggered by an airflow greater than 19 L/min by a user and the drug foil substrate was heated at ≈310-323° C.

After its use, the devices were opened, and visually inspected for the presence of apomorphine HCl deposited in the internal side of the housing (green tint). Then, the internal side of the housing of all the devices was rinsed at least 3 times with methanol to extract all the apomorphine HCl deposited. The solution was analysed as in Method 1.

The following table shows the results of the testing of the apomorphine HCl deposition on the internal walls. The devices showing by visual observations Apomorphine HCl deposition on the internal airway walls (green tint) were tested for Apomorphine HCl content. Min (Minimum), Max (maximum) and Av (Average) values of Apomorphine HCl content are detailed in mg and the % of deposited Apomorphine HCl are calculated over the target emitted dose.

| | | | | Apomorphine HCl deposition on | |
|---|---|---|---|---|---|
| Target | Devices | Green tint | | devices with green tint | |
| ED | No | No | % | mg (range) | % (range) |
| 1.0 mg | 43 | 17 | 40 | 0.066 (0.005-0.857) | 6.6 (0.5-85.7) |
| 2.0 mg | 25 | 6 | 24 | 0.104 (0.007-1.550) | 5.2 (0.3-77.5) |

### Comparative Example 2: Emitted Dose (ED) on Prior Art Devices Actuated by a Testing Apparatus

2 mg (for achieving a target ED of 1 mg) or 4 mg (for achieving a target ED of 2 mg) of apomorphine HCl was coated on the foil substrate of Devices A. The devices were tested as in Method 2 triggered at a 30 mL/min airflow and the drug foil substrate was heated to about 315° C.

The following table shows the results of the emitted dose measured, its Relative Standard Deviation (RSD) and the % emitted dose are calculated over the target emitted dose.

| | | emitted dose | | | | | |
|---|---|---|---|---|---|---|---|
| | | | | | | | Av |
| Target | Devices | Min | Min | Max | Max | Av | % |
| ED | No | mg | % | mg | % | mg | (RSD) |
| 1.0 mg | 10 | 0.695 | 69.5 | 1.688 | 168.8 | 1.068 | 106.8 |
| | | | | | | | (28.2) |
| 2.0 mg | 10 | 1.365 | 68.2 | 2.692 | 134.6 | 1.908 | 95.4 |
| | | | | | | | (19.2) |

Min: Minimum. Max: maximum. Av: Average

### Comparative Example 3: Emitted Dose (ED) Versus Temperature Calibration of Prior Art Devices

A calibration of the ED versus temperature (T° C.) calibration for Devices A coated with 4 mg (for a target ED 1 mg) apomorphine HCl on the drug foil was carried out. The devices were tested as in Method 2.

As shown in FIG. 3, for the Devices A (prior art configuration), to achieve an 80-120% of the target ED the electrically heated foil substrate must be heated to 310-323° C. at an air flow rate of 30 L/min.

### Comparative Example 4: Purity and Emitted Dose (ED) Versus Temperature Calibration of Prior Art Devices

The foil substrates of Devices A were coated with 1.0 mg of apomorphine HCl and tested as in Method 2 at different drug foil temperatures (2 devices per temperature point except 310° C. where 4 devices were used). The devices are triggered at a 30 mL/min airflow. The results of the purity of the aerosol shown in the next table:

| Drug foil (° C.) | TAerosol purity (%) |
|---|---|
| 260 | 98.4 ± 0.6 |
| 290 | 96.4 ± 0.9 |
| 310 | 95 ± 1 |
| 320 | 90.9 ± 0.8 |

The results clearly show that purity of the apomorphine HCl aerosol decreases as the temperature increases.

### Example 1: Emitted Dose (ED) on a Device Actuated by a Testing Apparatus

2 mg (for a target ED of 1 mg) or 4 mg (for a target ED of 2 mg) of apomorphine HCl was coated on the foil substrate of Devices B. The devices were tested as in Method 2 and the drug foil substrate was heated at ˜ 255-265° C. The devices are triggered at a 30 mL/min airflow.

The following table shows the results of the emitted dose measured, its Relative Standard Deviation (RSD) and the % emitted dose are calculated over the target emitted dose.

| | | emitted dose | | | | | |
|---|---|---|---|---|---|---|---|
| | | | | | | | Av |
| Target | Devices | Min | Min | Max | Max | Av | % |
| ED | No | mg | % | mg | % | mg | (RSD) |
| 1.0 mg | 10 | 0.891 | 89.1 | 1.241 | 124.1 | 1.028 | 103 |
| | | | | | | | (10) |
| 2.0 mg | 10 | 1.744 | 87.1 | 2.325 | 116.1 | 2.003 | 100 |
| | | | | | | | (8) |

Min: Minimum. Max: maximum. Av: Average.

When Example 1 is compared with Comparative Example 2 it is shown that the dispersion of the emitted dose (ED) is lower and more centerd in the target value in Example 1, and, contrary to Comparative Example 3, this is achieved using a lower temperature in Example 1, which allows for an increase in the purity of the aerosol.

### Example 2: CFD Modelling of Prior Art Cartridges and Cartridges

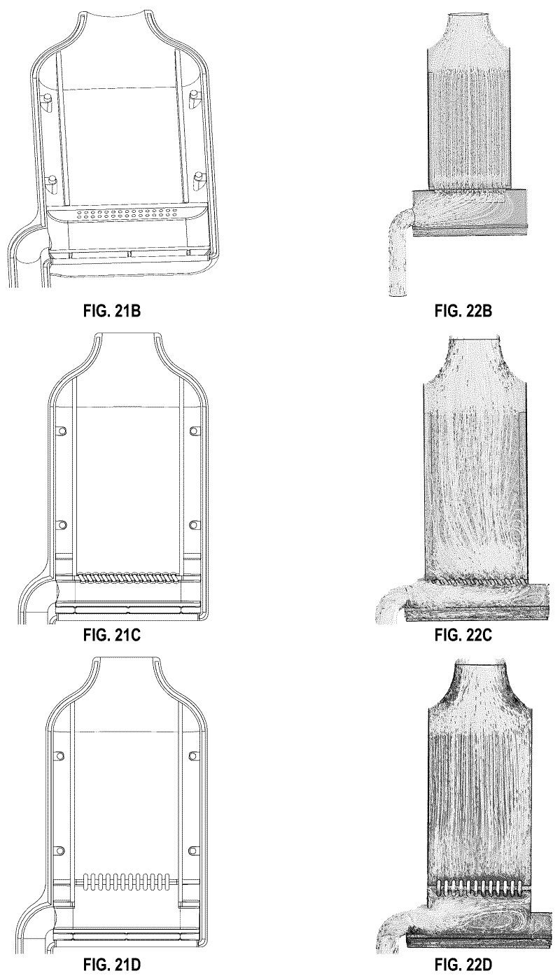

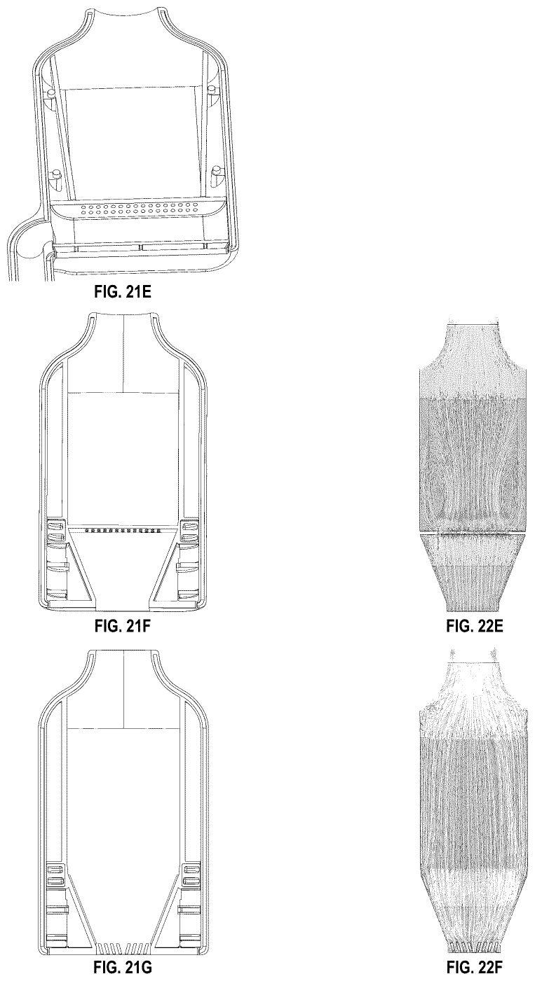

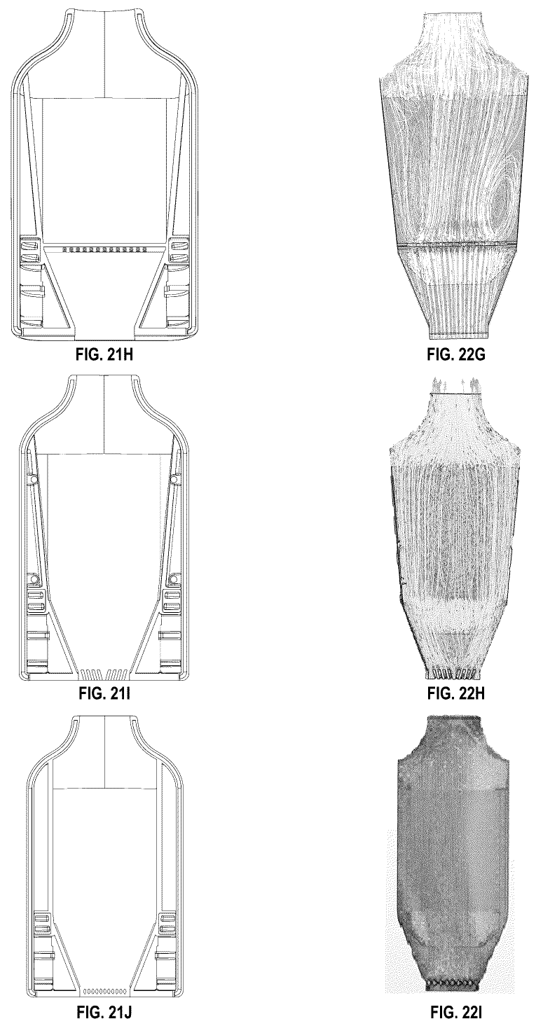

Computational Fluid Dynamics (CFD) of devices C-M (see FIGS. 21A-21K) has been created using the Simulia® 3D software under the 3DEXPERIENCE® platform version R2023x.HotFix0.10. The software uses Navier-Stokes equations solver to solve the mathematical model based on partial differential equations that describe the fluid flow. The CFD software Simulia® 3D experience solves the Mathematical models with boundary conditions specific to our problem.

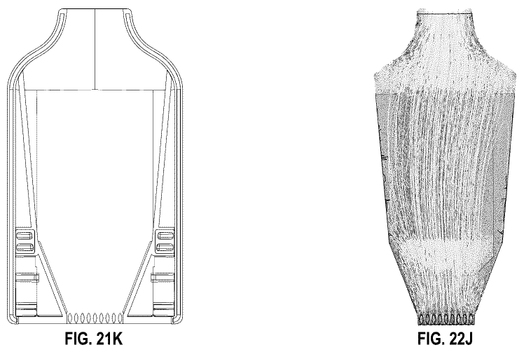

FIGS. 22A-22K show velocity plots of devices C-M respectively. The streamlines (the white lines) indicate the direction of the airflow, the denser the number of lines the higher the air flow velocity. (variability or more interesting or less uniformity)

FIG. 22A illustrates the velocity plot of Device C depicting the high velocity and turbulence provoked by the narrow and sided air inlet. The diverting and directing structures allow for a more uniform, slower, and less turbulent airflow in the whole disposable cartridge. Compare with the CFD modelling in 22F where the perforated bulkhead introduces turbulence.

FIG. 22C illustrates the velocity plot of Device E depicting that the diverting structures orientate the streamlines towards the air outlet and slightly reduces the airflow velocity. Compare with the CFD modelling in 22F or 22H where the perforated bulkhead introduces turbulence.

FIG. 22D illustrates the velocity plot of Device F depicting that the straightening structures orientate the streamlines towards the air outlet, make the airflow more uniform and slightly reduces the airflow velocity in the whole cartridge. Compare with the CFD modelling in 22F or 22H where the perforated bulkhead introduces turbulence.

FIG. 22F illustrates the velocity plot of Device H depicting that the bulkhead introduces turbulence at each side of the cartridge.

FIG. 22G illustrates the velocity plot of Device I depicting that the directing features are able to uniformly distribute the airflow in the whole cartridge. Compare with the CFD modelling in 22F or 22H where the perforated bulkhead introduces turbulence.

FIG. 22H illustrates the velocity plot of Device J depicting that the bulkhead introduces turbulence and does not homogeneously distribute the airflow in the whole cartridge.

FIG. 22I illustrates the velocity plot of Device K depicting that the directing structures allow for a uniform distribution of the airflow in the whole disposable cartridge. Compare with the CFD modelling in 22F or 22H where the perforated bulkhead introduces turbulence.

FIG. 22J illustrates the velocity plot of Device L depicting that the straightening structures do not introduce turbulence in the cartridge. Compare with the CFD modelling in 22F or 22H where the perforated bulkhead introduces turbulence.

FIG. 22K illustrates the velocity plot of Device M (with the straightening structures having aerodynamic elliptical shape) depicting showing a quite homogeneous airflow and less turbulence compared with the CFD modelling in 22F or 22H having a perforated bulkhead.

### THE REFERENCE NUMBERS IN THE FIGURES MEAN

100: Handheld controller.
101: Batteries.
102: Microcontroller.
103: Air inlet controller extension.
200: Single dose disposable cartridge.
202: Air outlet adapted as a mouthpiece.
203: Airway defined by the internal walls.
204: Drug foil substrate support.
205: Drug foil substrate.
206: Perforated thin bulkhead.
207: Solid drug film
220: Air inlet.
221: Parallel straight internal opposite walls.
221a: Parallel-convex internal opposite walls.
221b: Parallel-concave internal opposite walls.
222: Angled straight internal opposite walls.
222a: Angled-convex internal opposite walls.
222b: Angled-concave internal opposite walls.
223: Aerosol axis.
224: Airflow diverting structure.
224C: Cavities of the airflow diverting structure.
224CH: Channels of the airflow diverting structure.
224SL: Slit of the airflow diverting structure.
225: Airflow straightening structure.
225C: Cavities of the airflow straightening structure.
225CH: Channels of the airflow straightening structure.
225SL: Slit of the airflow straightening structure.
226: Airflow directing structures.
226CHL and 226CHR: Channels of the airflow directing features
301: Data interface between the handheld controller (100) and disposable cartridge (200).
302: Electric interface between the handheld controller (100) and disposable cartridge (200).
α: Angle formed by the centerlines of the channels (225CH) of the airflow straightening structures (225) with the aerosol axis (223).
β: Angle formed by the centerlines of the channels (224CH) of the airflow diverting structures (224) in respect of the ray of the aerosol axis (223) pointing towards the upstream (U_{A}) in the sector containing the air inlet (220).
δ: Angle formed by two internal opposite walls (222, 222a, 222b).
γ: Angle formed by the airflow directing features (226) with the Z-plane; where the vertex of said angle γ is located upstream the drug foil substrate.
C: Crossbar of a three-dimensional (3D) H, right tack symbol (├) or left tack symbol (┤).
E: Possible location of the electronics.
D_{A}: Downstream of the aerosol axis (223).
U_{A}: Upstream of the aerosol axis (223).
PCB: Printed Circuit Board.
Pf: Projection of the drug foil substrate (205) on the aerosol axis (223).
S: Stems of a three-dimensional (3D) H, right tack symbol (├) or left tack symbol (┤).
Wp: A wall of a pentagon.
Wh: A wall of a hexagon.

1. A medical device for generating a drug condensation aerosol by thermal vaporization of a drug, the medical device comprising:
a. an airway defined by internal walls;
b. an air inlet at one end of the airway;
c. an air outlet, configured as a mouthpiece, the air outlet located at another end of the airway;
d. a drug foil substrate having an impermeable surface placed within the airway;
e. a drug foil substrate support;
f. a solid drug film coated on the drug foil substrate;
g. an aerosol axis defined by at a geometrical center of the solid drug film and a geometrical center of the air outlet, wherein a downstream direction (D_{A}) goes from the geometrical center of the solid drug film to the geometrical centre of the air outlet and an upstream direction (U_{A}) is the opposite direction;
h. a drug foil substrate heating circuit configured to vaporize at least part of the drug in the solid drug film to produce condensation aerosol particles, wherein the drug foil substrate heating circuit comprises at least one battery, and
i. one or more aerosol particle delivery modifiers selected from the group consisting of:
I. one or more airflow diverting structures which comprise a structure placed upstream of the drug foil substrate comprising a plurality of channels, wherein the centerlines of the channels define an angle β from 100° to 170° with the ray of the aerosol axis in the upstream (U_{A}) direction measured in the sector containing the air inlet;
II. one or more airflow straightening structures which comprise a structure placed upstream of the drug foil substrate comprising a plurality of channels, wherein the centerlines of the channels define an angle α from −10° to 10° with the aerosol axis;
III. internal walls comprising two internal opposite walls which define an angle δ of 5° to 20° in at least 80% of its portion comprised within the projection (Pf) of the drug foil substrate on the aerosol axis; wherein the vertex of said angle δ is located in the upstream (U_{A}) direction; and
IV. one or more airflow directing structures which comprise a structure placed upstream of the drug foil substrate comprising at least two sets of channels, one set at each side of the Z-plane, wherein centerlines of each set of channels define an angle γ from 5° to 20° with the Z-plane; and wherein the vertex of said angle γ is located upstream of the drug foil substrate.

2. The device of claim 1, wherein the drug foil substrate heating circuit comprises an electrical heater.

3. The device of claim 2, further comprising a disposable cartridge and a handheld controller, wherein the disposable cartridge is configured to be connected to the handheld controller, the disposable cartridge comprises features a) to g), and i), and the handheld controller comprises feature h).

4. A disposable cartridge configured to be connected to a handheld controller, the handheld controller comprising: an airway defined by internal walls; an air inlet at one end of the airway; an air outlet, configured as a mouthpiece, the air outlet located at another end of the airway; a drug foil substrate having an impermeable surface placed within the airway; a drug foil substrate support; a solid drug film coated on the drug foil substrate; an aerosol axis defined by at a geometrical center of the solid drug film and a geometrical center of the air outlet, wherein a downstream direction (D_{A}) goes from the geometrical center of the solid drug film to the geometrical center of the air outlet and an upstream direction (U_{A}) is the opposite direction; and one or more aerosol particle delivery modifiers selected from the group consisting of:
a. one or more airflow diverting structures which comprise a structure placed upstream of the drug foil substrate comprising a plurality of channels, wherein the centerlines of the channels define an angle β from 100° to 170° with the ray of the aerosol axis in the upstream (U_{A}) direction measured in the sector containing the air inlet;
b. one or more airflow straightening structures which comprise a structure placed upstream of the drug foil substrate comprising a plurality of channels, wherein the centerlines of the channels define an angle α from −10° to 10° with the aerosol axis;
c. internal walls comprising two internal opposite walls which define an angle δ of 5° to 20° in at least 80% of its portion comprised within the projection (Pf) of the drug foil substrate on the aerosol axis; wherein the vertex of said angle δ is located in the upstream (U_{A}) direction; and
d. one or more airflow directing structures which comprise a structure placed upstream of the drug foil substrate comprising at least two sets of channels, one set at each side of the Z-plane, wherein centerlines of each set of channels define an angle γ from 5° to 20° with the Z-plane; and wherein the vertex of said angle γ is located upstream of the drug foil substrate.

5. The disposable cartridge of claim 4, wherein the aerosol particle delivery modifiers comprise one or more airflow diverting structures which comprise a structure placed upstream of the drug foil substrate comprising a plurality of channels, wherein the centerlines of the channels define an angle β from 100° to 170° with the ray of the aerosol axis in the upstream (U_{A}) direction measured in the sector containing the air inlet.

6. The disposable cartridge of claim 4, wherein the aerosol particle delivery modifiers comprise one or more airflow straightening structures which comprise a structure placed upstream of the drug foil substrate comprising a plurality of channels, wherein the centerlines of the channels define an angle α from −10° to 10° with the aerosol axis.

7. The disposable cartridge of claim 4, wherein the aerosol particle delivery modifiers comprise. internal walls comprising two internal opposite walls which define an angle δ of 5° to 20° in at least 80% of its portion comprised within the projection (Pf) of the drug foil substrate on the aerosol axis; wherein the vertex of said angle δ is located in the upstream (U_{A}) direction.

8. The disposable cartridge of claim 4, wherein the aerosol particle delivery modifiers comprise one or more airflow directing structures which comprise a structure placed upstream of the drug foil substrate comprising at least two sets of channels, one set at each side of the Z-plane, wherein centerlines of each set of channels define an angle γ from 5° to 20° with the Z-plane; and wherein the vertex of said angle γ is located upstream of the drug foil substrate.

9. The disposable cartridge of claim 4, wherein the aerosol particle delivery modifiers comprise:
a. one or more airflow diverting structures which comprise a structure placed upstream of the drug foil substrate comprising a plurality of channels, wherein the centerlines of the channels define an angle β from 100° to 170° with the ray of the aerosol axis in the upstream (U_{A}) direction measured in the sector containing the air inlet;
b. one or more airflow straightening structures which comprise a structure placed upstream of the drug foil substrate comprising a plurality of channels, wherein the centerlines of the channels define an angle α from −10° to 10° with the aerosol axis; and
c. one or more airflow directing structures which comprise a structure placed upstream of the drug foil substrate comprising at least two sets of channels, one set at each side of the Z-plane, wherein centerlines of each set of channels define an angle γ from 5° to 20° with the Z-plane; and wherein the vertex of said angle γ is located upstream of the drug foil substrate.

10. The disposable cartridge of claim 4, wherein the aerosol axis crosses within the central region that comprises half of the channels of the most upstream of the one or more airflow diverting structures and the one or more airflow straightening structures.

11. The disposable cartridge of claim 4, wherein the channels of the one or more airflow diverting structures extend from 0.7 to 5 mm measured in the centerline direction.

12. The disposable cartridge of claim 4, wherein the one or more airflow diverting structures comprise cavities upstream and/or downstream of the channels, wherein the channels and the cavities are:
a. H shaped,
b. right tack shaped (├), or
c. left tack shaped (┤); and
the channels being the crossbar of the H, ├ or ┤; the cavities being the stems of the H, ├ or ┤; and the cavities extending up to 100% of an internal height of the device the closest of just downstream or upstream of the one or more airflow diverting structures.

13. The disposable cartridge of claim 4, wherein the channels of the one or more airflow straightening structures extend from 0.9 to 20 mm long measured in the centerline direction.

14. The disposable cartridge of claim 4, wherein the one or more airflow straightening structures comprise cavities upstream and/or downstream of the channels, wherein the channels and the cavities are
a. H shaped,
b. right tack shaped (├), or
c. left tack shaped (┤); and
the channels being the crossbar (C) of the H; ├ or ┤, and the cavities being the stems(S) of the H, ├ or ┤; and the cavities extending up to 100% high of the internal height of the device the closest of just downstream or upstream of the one or more airflow straightening structures.

15. The disposable cartridge of claim 4, wherein the internal opposite walls are straight, convex or concave.

16. The disposable cartridge of claim 4, wherein the angle γ ranges from 10° to 20°.

17. The disposable cartridge of claim 4, wherein any of the diverting channels, straightening channels and/or airflow directing channels have an elliptical shape.

18. The disposable cartridge for use in therapy according to claim 4, wherein when the drug in the solid drug film is:
a. loxapine or its pharmaceutically acceptable salts, and a condition or episode is agitation, comprising:
I. rapidly control mild to moderate agitation in adults with schizophrenia or bipolar disorder, or
II. acute agitation associated with schizophrenia or bipolar disorder in adults;
b. alprazolam, estazolam or its pharmaceutically acceptable salts, a condition or episode is epilepsy, wherein epilepsy comprises seizures;
c. fentanyl or its pharmaceutically acceptable salts, a condition or episode is breakthrough pain;
d. zaleplon, almorexant or its pharmaceutically acceptable salts, a condition or episode is a sleep disorder comprising:
I. middle of the night awakening, or
II. middle of the night insomnia;
e. apomorphine, pergolide, ropinirole, pramipexole, or its pharmaceutically acceptable salts, a condition or episode is Parkinson's disease, off-episodes in Parkinson's disease, and/or idiopathic Parkinson's disease;
f. granisetron, ondansetron, palonosetron or its pharmaceutically acceptable salts, a condition or episode is:
I. nausea,
II. vomiting or
III. cyclic vomiting syndrome;
g. nicotine or its pharmaceutically acceptable salts including nicotine meta-salicylate, a condition or episode is nicotine craving and/or effecting cessation of smoking; or
h. ropinirole, pramipexole, or rotigotine, and a condition or episode is restless legs syndrome.
